SEQUENCE LISTING (25 sequences)

SEQ ID NO: 1 - protein, 452 aa, Artificial Sequence
  Amino acid sequence of anti-EGFR heavy chain
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 213 aa, Artificial Sequence
  Amino acid sequence of anti-EGFR light chain
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGA

SEQ ID NO: 3 - protein, 21 aa, Artificial Sequence
  Amino acid sequence of OmpA signal peptide
MKKTAIAIAVALAGFATVAQA

SEQ ID NO: 4 - protein, 119 aa, Artificial Sequence
  Amino acid sequence of Heavy chain Variable region
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 5 - protein, 102 aa, Artificial Sequence
  Amino acid sequence of Heavy chain Constant region 1
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKS

SEQ ID NO: 6 - protein, 107 aa, Artificial Sequence
  Amino acid sequence of Light chain Variable region
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 7 - protein, 106 aa, Artificial Sequence
  Amino acid sequence of Light chain Constant region
RTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGA

SEQ ID NO: 8 - DNA, 63 bp, Artificial Sequence
  Nucleotide sequence of OmpA signal peptide
atgaaaaagacagctatcgcgattgcagtggcactggctggtttcgctaccgtagcgcaggcc

SEQ ID NO: 9 - DNA, 357 bp, Artificial Sequence
  Nucleotide sequence of Heavy chain Variable region
caagtccaactgaaacaatcgggtccgggtctggtccaaccgtcccaatcactgagcatcacctgtaccgtgtcgggcttctcgctgaccaattatggtgtgcattgggttcgtcagagtccgggcaaaggtctggaatggctgggcgttatttggtccggcggtaataccgattacaacaccccgtttacgagtcgcctgtccatcaataaagacaactcgaaaagccaggtgtttttcaaaatgaattcactgcaatcgaacgataccgcgatttattactgcgcacgtgctctgacgtattacgactatgaatttgcctactggggccagggtaccctggtgacggttagcgcg

SEQ ID NO: 10 - DNA, 306 bp, Artificial Sequence
  Nucleotide sequence of Heavy chain Constant region 1
gcctctaccaaaggtccgagcgttttcccgctggcaccgagctctaaatctaccagtggcggtacggcagctctgggctgtctggtgaaagattattttccggaaccggtcaccgtgagttggaattccggtgcactgaccagtggcgtccacacgttcccggctgtgctgcagagttccggtctgtatagcctgtcatcggtggttaccgttccgagctctagtctgggcacccaaacgtacatttgcaatgtcaaccataaaccgagcaacacgaaagttgataaacgtgtcgaaccgaaatca

SEQ ID NO: 11 - DNA, 321 bp, Artificial Sequence
  Nucleotide sequence of Light chain Variable region
gatattctgctgacccagagcccggtgatcctgagtgtttccccgggcgaacgtgtgtcattttcgtgtcgcgcgagccagtctattggtaccaatatccactggtatcagcaacgtacgaacggctctccgcgcctgctgattaaatacgccagtgaatccatttcaggcatcccgagccgcttttcgggcagcggttctggcaccgatttcacgctgagtattaactccgtggaatcagaagatatcgcagactattactgccagcaaaacaataactggccgaccacgtttggtgctggcaccaaactggaactgaaa

SEQ ID NO: 12 - DNA, 318 bp, Artificial Sequence
  Nucleotide sequence of Light chain Constant region
cgtacggtggcggccccgagtgtttttatcttcccgccgtccgatgaacagctgaaatcgggtaccgccagcgttgtctgtctgctgaataacttctatccgcgcgaagcaaaagtccagtggaaagtggacaatgctctgcagtcgggcaacagccaagaaagcgtgaccgaacaagatagtaaagactccacgtactcactgtcctcaaccctgacgctgagcaaagcggattatgaaaaacacaaagtgtacgcctgcgaagttacccatcaaggtctgagtagcccggttacgaaatcattcaatcgtggtgcc

SEQ ID NO: 13 - DNA, 37 bp, Artificial Sequence
  Forward primer of Heavy chain Variable region
cgcccatggccaaaaagacaacagctatcgcgattgc

SEQ ID NO: 14 - DNA, 37 bp, Artificial Sequence
  Reverse primer of Heavy chain Constant region 1
atgcggccgcaagcttctatgatttcggttcgacacg

SEQ ID NO: 15 - DNA, 57 bp, Artificial Sequence
  Forward primer of Light chain Variable region
aaggagatatacatatgaaaaagacagctatcgcgattgcagtggcactggctggtt

SEQ ID NO: 16 - DNA, 37 bp, Artificial Sequence
  Reverse primer of Light chain Constant region
ctttaccagactcgagctaggcaccacgattgaatga

SEQ ID NO: 17 - protein, 105 aa, Artificial Sequence
  Amino acid sequence of Heavy chain Constant region 1 + CDK
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDK

SEQ ID NO: 18 - protein, 108 aa, Artificial Sequence
  Amino acid sequence of Light chain Constant retion +EC
RTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGAEC

SEQ ID NO: 19 - DNA, 315 bp, Artificial Sequence
  Nucleotide sequence of Heavy chain Constant region 1 +CDK
gcctctaccaaaggtccgagcgttttcccgctggcaccgagctctaaatctaccagtggcggtacggcagctctgggctgtctggtgaaagattattttccggaaccggtcaccgtgagttggaattccggtgcactgaccagtggcgtccacacgttcccggctgtgctgcagagttccggtctgtatagcctgtcatcggtggttaccgttccgagctctagtctgggcacccaaacgtacatttgcaatgtcaaccataaaccgagcaacacgaaagttgataaacgtgtcgaaccgaaatcatgcgataaa

SEQ ID NO: 20 - DNA, 324 bp, Artificial Sequence
  Nucleotide sequence of Light chain Constant region +EC
cgtacggtggcggccccgagtgtttttatcttcccgccgtccgatgaacagctgaaatcgggtaccgccagcgttgtctgtctgctgaataacttctatccgcgcgaagcaaaagtccagtggaaagtggacaatgctctgcagtcgggcaacagccaagaaagcgtgaccgaacaagatagtaaagactccacgtactcactgtcctcaaccctgacgctgagcaaagcggattatgaaaaacacaaagtgtacgcctgcgaagttacccatcaaggtctgagtagcccggttacgaaatcattcaatcgtggtgccgaatgc

SEQ ID NO: 21 - DNA, 46 bp, Artificial Sequence
  Reverse primer of Heavy chain Constant region 1 +CDK
atgcggccgcaagcttctatttatcgcatgatttcggttcgacacg

SEQ ID NO: 22 - DNA, 43 bp, Artificial Sequence
  Reverse primer of Light chain Constant region +EC
ctttaccagactcgagctagcattcggcaccacgattgaatga

SEQ ID NO: 23 - protein, 111 aa, Artificial Sequence
  Amino acid sequence of Heavy chain Constant region 1 +CDK+THTCAA
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCAA

SEQ ID NO: 24 - DNA, 333 bp, Artificial Sequence
  Nucleotide sequence of Heavy chain Constant region 1 +CDK +THTCAA
gcctctaccaaaggtccgagcgttttcccgctggcaccgagctctaaatctaccagtggcggtacggcagctctgggctgtctggtgaaagattattttccggaaccggtcaccgtgagttggaattccggtgcactgaccagtggcgtccacacgttcccggctgtgctgcagagttccggtctgtatagcctgtcatcggtggttaccgttccgagctctagtctgggcacccaaacgtacatttgcaatgtcaaccataaaccgagcaacacgaaagttgataaacgtgtcgaaccgaaatcatgcgataaaacccatacctgcgcggcg

SEQ ID NO: 25 - DNA, 51 bp, Artificial Sequence
  Reverse primer of Heavy chain Constant region 1 +CDK+THACAA
cgcaagcttctacgccgcgcaggtatgggttttatcgcatgatttcggttc

(19) United States
(12) Patent Application Publication
(10) Pub. No.: US 2017/0145101 A1
(43) Pub. Date: May 25, 2017

(54) Fab FRAGMENT SPECIFICALLY BINDING TO EGFR

(71) Applicant: SHIN-IL PHARMACEUTICAL CO., LTD.

(72) Inventors: Young Pil KIM, Gyeonggi-do (KR); Hye Rim KIM, Gyeonggi-do (KR); Hee Jung YOO, Gyeonggi-do (KR); Seong Hwan LEE, Gyeonggi-do (KR); Tai Geun JUNG, Gyeonggi-do (KR); Jong Ryul HA, Seoul (KR)

(21) Appl. No.: 14/914,608

(22) PCT Filed: Dec. 7, 2015
(86) PCT No.: PCT/KR2015/013322

§ 371 Date: Feb. 25, 2016

(30) Foreign Application Priority Data
Apr. 23, 2015 (KR) 10-2015-0057262
Sep. 8, 2015 (KR) 10-2015-0126894

(51) Int. Cl. C07K 16/28

(52) U.S. Cl. CPC C07K 16/2863; C07K 2317/55; C07K 2317/56; C07K 2317/522; C07K 2317/40; C07K 2317/51; C07K 2317/515; C07K 2317/92; A61K 2039/505

(57) ABSTRACT

The present invention relates to a fragment antigen-binding (Fab) fragment specifically binding to epidermal growth factor receptor (EGFR), an expression construct for preparing the Fab fragment, a method for preparing the Fab fragment, and a pharmaceutical composition containing the Fab fragment. The Fab fragment to EGFR of the present invention is smaller than the antibody, and thus can favorably permeate into tissues or tumors and can be prepared in bacteria, resulting in low production costs. Furthermore, the Fab fragment to EGFR of the present invention has an increased in vivo half-life through pegylation.

### FIELD

The present invention was made with the support of the Ministry of Trade, Industry and Energy, Republic of Korea, under Project No. A004600265, which was conducted in the program titled “Chung-Cheong Province Economic Bloc Lead Business Development Project” in the project named “Development of Targeted Anticancer Agent Anti-EGFR Bio Vector” by SINIL Pharmaceutical Co., Ltd., under management of the Chungcheong Institute for Regional Program Evaluation, during the period of 1 Aug. 2012 to 30 Apr. 2015.

The present application claims priorities from Korean Patent Application No. 10-2015-0057262 filed with the Korean Intellectual Property Office on 23 Apr. 2015 and Korean Patent Application No. 10-201 5-0126894 filed with the Korean Intellectual Property Office on 8 Sep. 2015, the disclosures of which are hereby incorporated herein by reference into this application.

The present invention relates to a fragment antigen-binding (Fab) fragment specifically binding to an epidermal growth factor receptor (EGFR).

### BACKGROUND

Epidermal growth factor receptor (EGFR; HER1), which is one member of the receptor HER family existing on cell surfaces, plays an important role in cell growth and death by binding with ligands, such as EGF, TGF-α, and epiregulin. Particularly, EGFR has attracted with respect to cancers through the reports that, as a result of research through immunostaining assay, many kinds of cancer cells show an increased EGFR expression, and such an increased EGFR expression is also closely related to prognosis (Nicholson, R. I. et al., Eur. J. Cancer., 37: S9-15 (2001); and Yewale, C, et al., Biomaterials, 34:8690-707 (2013)). So, there have been attempts to treat cancers by suppressing EGFR signals, and cetuximab, which is an EGFR blocking antibody, and gefitinib (Iressa®) or erlotinib (Tarceva®), which is a low-molecular weight EGFR tyrosin kinase inhibitor (EGFR-TK1), have been developed and used in clinical trials.

EGFR-TK1 targets EGFR like cetuximab, but is efficacious for lung cancer unlike cetuximab. EGFR-TK1 is less effective on the cancers that are known to have a close relation between the EGFR expression and the prognosis, but is effective on the lung cancer having a very weak relation therebetween, suggesting that there is another mechanism irrelevant to a signal difference depending on the EGFR expression level. In order to allow therapeutic antibodies to have cytotoxic effects on cancer cells, several action mechanisms are used in combination, and in most cases, effects are shown by immune systems through antibody-dependent cellular cytotoxicity (ADCC) or complement-dependent cytotoxicity (CDC).

Antibodies are structurally classified into five subfamilies: IgG, IgA, IgM, IgD, and IgE, based on the difference in the constant region (Fc) of the heavy chain. It has been known that, among the antibody isotypes, IgG1 and IgG3 have strong ADCC and CDC functions, IgG2 has no ADCC function and a weak CDC function, and IgG4 has a weak ADCC function but no CDC function. In cases of antibodies that have been developed in the anticancer field, the IgG1 isotype known to have high ADCC and CDC functions has been developed most frequently.

However, IgG1 type and IgG2 type antibody therapeutic agents have been developed and made commercially available in cases of EGFR unlike other anticancer targets, and this may disprove that the neutralizing activity due to antibodies is a main action in targeting EGFR.

The EGFR exists in the form of a tethered monomer or in the form of an untethered monomer, which is an open-type structure, and then, when EGF binds to EGFR, a dimer form thereof is generated to activate a kinase, resulting in the transduction of signals. The therapeutic antibody, cetuximab, binds to EGFR instead of ligands, to suppress kinase activity and downstream signals. As a result, cell growth is inhibited and cell death is induced, and such binding suppresses the activation of receptors and blocks the resultant signaling pathways, resulting in reducing the infiltration of tumor cells into normal tissues and the proliferation of tumors into new sites. In addition, it is determined that cetuximab overall inhibits the proliferation of tumors by inhibiting the ability of tumor cells to restore damage due to chemotherapy and radiotherapy and inhibiting angiogenesis in tumors.

There are also reports that, as a proof that the therapeutic mechanism of antibodies targeting EGFR results from the inhibition of EGFR signaling, cetuximab and panitumumab are influenced by genetic mutation of KRAS, which is a downstream signaling material of EGFR (Lièvre, A. et al., Cancer Res., 66:3992-5 (2006); and Karapetis, C. S. et al., N. Engl. J. Med., 359:1757-65 (2008)). This KRAS mutation is found in 40-45% of colorectal cancers, and such drugs are used in cases where there is determined to be no mutation when the genetic mutation of “KRAS” is checked through biomarker tests for cancer patients.

Erbitux® is used together with chemotherapies, and this was developed as an EGFR chimeric antibody for treating head and neck cancer and colorectal cancer by ImClone systems. ImClone systems and Bristol-Myers Squibb have the rights for Erbitux® in North America, and Merck KGaA (Germany) has the rights for Erbitux® in areas other than North America, Erbitux® was authorized by the U.S. Food and Drug Administration in February 2004. Meanwhile, the patent right for cetuximab expired in 2015, and biosimilars thereof are currently being developed domestically. Various therapeutic antibodies related to EGFR have been developed and authorized abroad, or are currently being clinically tested, and anticancer therapeutic agents targeting EGFR are actively being developed.

Throughout the entire specification, many papers and patent documents are referenced and their citations are represented. The disclosure of the cited papers and patent documents are entirely incorporated by reference into the present specification, and the level of the technical field within which the present invention falls, and the details of the present invention are explained more clearly.

### DETAILED DESCRIPTION

### Technical Problem

The present inventors have endeavored to prepare a Fab fragment which can be substituted for anticancer antibodies suppressing epidermal growth factor receptor (EGFR) signals. As a result, the present inventors have developed a Fab fragment which can be expressed in E. coli and specifically binds to EGFR, and verified an excellent binding affinity and anticancer effect thereof, and thus completed the present invention.

Therefore, the present invention has been made in view of the above-mentioned problems, and an aspect of the present invention is to provide a Fab fragment specifically binding to EGFR.

Another aspect of the present invention is to provide an expression construct for preparing the Fab fragment.

Still another aspect of the present invention is to provide a recombinant vector containing the expression construct.

Another aspect of the present invention is to provide host cells transformed with the recombinant vector.

Still another aspect of the present invention is to provide a method for preparing a Fab fragment to EGFR.

Another aspect of the present invention is to provide a pharmaceutical composition for preventing or treating cancer.

Other purposes and advantages of the present disclosure will become more obvious with the following detailed description of the invention, claims, and drawings.

### Technical Solution

In accordance with an aspect of the present invention, there is provided a fragment antigen-binding (Fab) fragment specifically binding to epidermal growth factor receptor (EGFR), the Fab fragment including:

(a) a heavy chain variable region (V_{H}) including an amino acid sequence of SEQ ID NO: 4;

(b) a heavy chain variable region 1 (C_{H1}) including an amino acid sequence of SEQ ID NO: 5;

(a) a light chain variable region (V_{L}) including an amino acid sequence of SEQ ID NO: 6; and

(d) a light chain constant region (C_{L}) including an amino acid sequence of SEQ ID NO: 7.

The present inventors have endeavored to prepare a Fab fragment which can be substituted for anticancer antibodies suppressing EGFR signals. As a result, the present inventors have developed a Fab fragment which can be expressed in E. coli and specifically binds to EGFR, and verified excellent binding affinity and anticancer effect thereof.

The Fab fragment of the present invention specifically binds to the EGFR.

Herein, the term “Fab fragment” refers to a fragment that retains an antigen-binding function, and has a structure with a heavy chain variable region (V_{H}), a heavy chain constant region 1 (C_{H1}), a light chain variable region (V_{L}), and a light chain variable region (C_{L}), and has one antigen-binding site. Fab′ is different from Fab in that the former has a hinge region including one or more cysteine residues at the C-terminal of the heavy chain C_{H1} domain. F(ab′)2 antibody is formed through a disulfide bond between the cysteine residues at the hinge region of Fab′, These Fab fragments may be obtained using proteases (for example, the whole antibody is digested with papain to obtain Fab fragments, or is digested with pepsin to obtain F(ab′)2 fragments), and may be, preferably, prepared by a genetic recombinant technique.

Herein, in order to improve production costs, considered to be a disadvantage in applying antibodies to the prevention or treatment of diseases, a Fab fragment is prepared by being expressed in E. coli, but not the whole antibody.

As used herein, the term “heavy chain” refers to the full-length heavy chain and fragments thereof, the full-length heavy chain including a variable region domain V_{H} that includes an amino acid sequence sufficient to provide specificity to an antigen, and three constant region domains, C_{H1}, C_{H2}, and C_{H3}.

The Fab fragment of the present invention is a Fab fragment including a heavy chain composed of V_{H} and C_{H1}.

In addition, as used herein, the term “light chain” refers to the full-length light chain and fragments thereof, the full-length light chain including a variable region domain V_{L} that includes an amino acid sequence sufficient to provide specificity to an antigen, and a constant region domain C_{L}.

The Fab fragment of the present invention is a Fab fragment including a light chain composed of V_{L} and C_{L}.

The Fab fragment of the present invention may include variants of amino acid sequences set forth in the appended sequence listing within the range in which the Fab fragment can specifically bind to EGFR. For example, the amino acid sequence of the Fab fragment may be altered to improve binding affinity and/or other biological characteristics of the Fab fragment. These alterations include, for example, deletion, insertion, and/or substitution of amino acid residues of the Fab fragment. Such amino acid alternations are made based on the relative similarity of the amino acid side-chain substitutions, for example, hydrophobicity, hydrophilicity, charge, size, or the like. An analysis of the size, shape, and type of the amino acid side-chain substituents may reveal that: arginine, lysine, and histidine are all positively charged residues; alanine, glycine, and serine are all similar in size; and phenylalanine, tryptophan, and tyrosine are all similar in shape. Therefore, on the basis of these considerations, arginine, lysine, and histidine; alanine, glycine, and serine; and phenylalanine, tryptophan, and tyrosine may be considered to be biologically functional equivalents. For introducing mutations, hydropathy indexes of amino acids may be considered. The hydropathy indexes are given to the respective amino acids depending on the hydrophobicity and charge: iso-leucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5) The hydrophobic amino acid indexes are very important in giving interactive biological functions of proteins. It is well known that amino acids with similar hydrophobic indexes need to be substituted with each other to retain similar biological activities. In cases where variations are introduced with reference to the hydrophobic indexes, the substitution is made between amino acids having a hydrophobic index difference within preferably ±2, more preferably ±1, and still more preferably ±0.5.

Meanwhile, it is also well known that the substitution between amino acids with similar hydrophilicity values results in proteins having equivalent biological activity. As disclosed in U.S. Pat. No. 4,554,101, the following hydrophilicity values are given to the respective amino acids, respectively: arginine (+3.0); lysine (+3.0); aspartate (+3.0±1); glutamate (+3.0 1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). In cases where the alternations are introduced with reference to the hydrophilic indexes, the substitution is made between amino acids having a hydrophilicity value difference within preferably ±2, more preferably ±1, and still more preferably ±0.5. Amino acid substitutions in the protein, without entirely altering molecular activity, are known in the art (H. Neurath, R. L. Hill, The Proteins, Academic Press, New York, 1979). The most common substitutions are substitutions between amino acid residues Ala/Ser, Val/Ile, Asp/Glu, Thr/Ser, Ala/Gly, Ala/Thr, Ser/Asn, Ala/Val, Ser/Gly, Thy/Phe, Ala/Pro, Lys/Arg, Asp/Asn, Leu/Ile, Leu/Val, Ala/Glu, and Asp/Gly. Considering the foregoing alterations with the biological equivalent activity, antibody or nucleic acid molecule encoding the antibody, of the present invention, are construed to also include sequences having substantial identity to the sequences set forth in the sequence listings. The substantial identity means that, when the sequence of the present invention and another optional sequence are aligned to correspond to each other as much as possible and the aligned sequences are analyzed using an algorithm that is commonly used in the art, the present sequence has at least 61%, more preferably at least 70%, still more preferably at least 80%, and most preferably at least 90% sequence identity. Methods of alignment of sequences for comparison are well known in the art. Various methods and algorithms for alignment are disclosed in: Smith and Waterman, Adv. Appl, Math. 2:482(1981); Needleman and Wunsch, J. Mol. Bio. 48:443(1970); Pearson and Lipman, Methods in Mol. Biol. 24: 307-31(1988); Higgins and Sharp, Gene 73:237-44(1988); Higgins and Sharp, CABIOS 5:151-3(1989); Corpet et al., Nuc, Acids Res. 16:10881-90(1988); Huang et al., Comp. Appl. BioSci. 8:155-65(1992); and Pearson et al., Meth. Mol. Biol. 24:307-31(1994). The NCBI Basic Local Alignment Search Tool (BLAST) (Altschul et al., J. Md. Biol. 215:403-10(1990)) is available from several sources, including the National Center for Biological Information (NCBI), and on the Internet, for use in connection with the sequence analysis programs blastp, blasm, blastx, tblastn, and tblastx, The BLAST is accessible at http://www.ncbi.nlm.nih.gov/BLAST/. The comparision of the sequence identity using this program can be confirmed at http://www,ncbi.nlm.nih.gov/BLAST/blast_help.html.

The Fab fragment specifically binding to EGFR, of the present invention, further includes an amino acid sequence for forming a disulfide bond between C_{H1} and C_{L}, in order to form a heterodimer of heavy and light chains.

According to an embodiment of the present invention, the C_{H1} further includes Cys-Asp-Lys at the C-terminal thereof (SEQ ID NO: 17).

According to another embodiment of the present invention, the C_{L} further includes Glu-Cys at the C-terminal thereof (SEQ ID NO: 18).

According to a specific embodiment of the present invention, the C_{H1} further includes Glu-Cys at the C-terminal thereof, and the Cys residue of Cys-Asp-Lys at the C-terminal of the C_{H1} is linked to the Cys residue of Glu-Cys at the C-terminal of the V_{L} via a disulfide bond.

The pegylation of the Fab fragment to EGFR is one of the main features in the present invention.

As used herein, the term “pegylation” refers to the conjugation of polyethylene glycol (PEG) to a target protein, that is, the Fab fragment to EGFR.

In order to solve a problem in that the in vivo drug sustainability and stability of the Fab fragment of the present invention are deteriorated, polyethylene glycol, which is a polymer that does not cause an immune response in vivo and thus has excellent biocompatibility, is conjugated to the Fab fragment. The polyethylene glycol is conjugated to a site in which the influence on drug activity can be minimized and the pegylation effect can be maximized, thereby minimizing deterioration in the efficacy of the Fab fragment. Since the molecular weight of the pegylated Fab fragment was increased, the penetration of the protein with respect to the filtering effect in the kidney due to glomerular filtration can be suppressed, so the loss of the protein is reduced. In addition, the degradation actions of in vivo proteases are inhibited through a stealth effect of the polyethylene glycol, so the in vivo half-life of the Fab fragment is increased. In addition, the steric hindrance of the polyethylene glycol prevents the approach of the in vivo proteases, thereby increasing stability against drugs and increasing solubility in aqueous solutions due to the hydrophilicity of the polyethylene glycol.

The C_{H1} of the Fab fragment to EGFR of the present invention is pegylated. For the pegylation of the Fab fragment, an amino acid sequence may be further linked.

According to an embodiment of the present invention, Thr-His-Thr-Cys-Ala-Ala may be further linked to Cys-Asp-Lys at the C-terminal of C_{H1} of the Fab fragment (SEQ ID NO: 23).

According to another embodiment of the present invention, the Cys residue in the Thr-His-Thr-Cys-Ala-Ala at the C-terminal of C_{H1} of the Fab fragment is pegylated.

As used herein, the term “polyethylene glycol (PEG)” refers to water-soluble poly(ethylene oxide). Typically, the PEG suitable in the present invention is expressed by the following structural formula: (OCH₂CH₂)ₙ (here, n is an integer of 2 to 4000). In addition, the PEG suitable in the present invention includes “CH₂CH₂O(CH₂CH₂O)ₙCH₂CH₂” and “(OCH₂CH₂)ₙO”. Furthermore, herein, the PEG includes structures having various terminal groups and “terminal capping” groups. For example, the terminal group includes maleimide.

According to an embodiment of the present invention, the PEG used in the pegylation has a molecular weight of 5-50 kDa.

According to another embodiment of the present invention, the PEG has a molecular weight of 18-38 kDa.

The Fab fragment of the present invention binds to one PEG molecule at 1:1.

The Fab fragment of the present invention has an excellent half-life through pegylation.

According to an embodiment of the present invention, the half-life of the Fab fragment in the mouse (Mus musculus) is 20-35 hours.

In accordance with another aspect of the present invention, there is provided an expression construct for preparing a fragment antigen-binding (Fab) fragment specifically binding to epidermal growth factor receptor (EGFR), the expression construct including:

(a) a heavy chain-expression construct including: (a-1) a heavy chain variable region (V_{H})-encoding nucleic acid molecule including a nucleotide sequence of SEQ ID NO: 9; and (a-2) a heavy chain constant region 1 (C_{H1})-encoding nucleic acid molecule including a nucleotide sequence of SEQ ID NO: 10; and

(b) a light chain-expression construct including : (b-1) a light chain variable region (V_{L})-encoding nucleic acid molecule including a nucleotide sequence of SEQ ID NO: 11: and (b-2) a light chain constant region (C_{L})-encoding nucleic acid molecule including a nucleotide sequence of SEQ ID NO: 12.

The expression construct for preparing the Fab fragment specifically binding to EGFR, of the present invention, is an expression construct for preparing the Fab fragment, and thus the descriptions of overlapping contents therebetween are omitted to avoid excessive complication of the present specification.

The heavy chain constant region 1 (C_{H1})-encoding nucleic acid molecule and the light chain constant region (CO-encoding nucleic acid molecule, which constitute the expression construct of the present invention, may further include a nucleotide sequence for forming a disulfide bond between the C_{H1} and the C_{L} and/or a nucleotide sequence for pegylation of the C_{H1} at the C-terminals of the nucleic acid molecules,

According to an embodiment of the present invention, the heavy chain constant region 1 (C_{H1})-encoding nucleic acid molecule is a nucleotide sequence of SEQ ID NO: 10, 19, or 24.

According to another embodiment of the present invention, the light chain constant region (C_{L})-encoding nucleic acid molecule is a nucleotide sequence of SEQ ID NO: 12 or 20.

As used herein, the term “nucleic acid molecule” refers to comprehensively including DNA (gDNA and cDNA) and RNA molecules, and the nucleotide as a basic constituent unit in the nucleic acid molecule includes naturally occurring nucleotides, and analogues with modified sugars or bases (Scheit, Nucleotide Analogs, John Wiley, New York(1980); Uhlman, and Peyman, Chemical Reviews, 90:543-584(1990)). The nucleic acid molecules encoding the heavy chain variable region and the light chain variable region of the Fab fragment of the present invention may be modified. The modification includes addition, deletion, or non-conservative substitution or conservative substitution.

The nucleic acid molecule encoding the antibody of the present invention is construed to also include a nucleotide sequence showing substantial identity to the foregoing nucleotide sequence. The term “substantial identity” means that, when the present nucleotide sequence and another nucleotide sequence are aligned to correspond to each other as much as possible and the aligned sequences are analyzed using an algorithm that is normally used in the art, the present nucleotide sequence has at least 80% sequence identity, preferably at least 90%, most preferably at least 95% sequence identity compared to another nucleotide sequence.

One of the main features of the present invention is that the Fab fragment to EGFR may be prepared through E. coli.

In the nucleic acid molecule, the nucleotide sequence encoding the Fab fragment to EGF R was converted to the favor of a host by reflecting the frequency of codon expression of E. coli, in order to express the Fab fragment in E. coli.

The expression construct produced in the preset invention is constructed to express desired genes in host cells. Generally, a promoter and a terminator are operatively linked to the upstream and the downstream of the expression construct, respectively.

As used herein, the term “promoter” refers to a DNA sequence that regulates the expression of a coding sequence or functional RNA. In the recombinant vector of the present invention, a target nucleotide sequence is operatively linked to the promoter.

As used herein, the term “operatively linked” refers to a functional linkage between a nucleic acid expression regulating sequence (e.g., a promoter sequence, a signal sequence, or an array at the binding site of a transcription control factor) and the another nucleic acid sequence, and the regulating sequence regulates the transcription and/or translation of the other nucleic acid sequence.

In accordance with still another aspect of the present invention, there is provided a recombinant vector including the expression construct.

The vector system of the present invention can be constructed through various methods known in the art, and a specific method thereof is disclosed in Sambrook et al., Molecular Cloning, A Laboratory Manual, Cold Spring Harbor Laboratory Press (2001), which is incorporated herein by reference.

The vector of the present invention may be typically constructed as a vector for cloning or a vector for expression. In addition, the vector of the present invention may be constructed by using a prokaryotic cell as a host. The vector of the present invention may be typically constructed as a vector for cloning or a vector for expression.

For example, in cases where the vector of the present invention is an expression vector and a prokaryotic cell is used as a host, it is general to contain strong promoters that can perform the transcription process (e. g., T7 promoter, tac promoter, lac promoter, lacUV5 promoter, Ipp promoter, pL λ promoter, pR λ promoter, rac5 promoter, amp promoter, recA promoter, SP6 promoter, and a trp promoter), a ribosomal binding site for initiating translation, and transcription/translation terminal sequences (terminator, e. g., T7 terminator, ADH1 terminator, T3 terminator, and TonB terminator). In cases where E. coli is used as host cells, the promoter and operator region for the tryptophan biosynthesis pathway (Yanofsky, C., J. Bacteriol., 158:1018-1024(1984)) and the leftward promoter from phage λ (pL λ promoter, Herskowitz, I. and Hagen, D., Ann. Rev. Genet., 14:399-445(1980)) may be used as regulating sequences.

On the other hand, the vector usable in the present invention may be constructed by manipulating a plasmid (e.g., pACYCDuet-1, pSC101, ColE1, pBR322, pUC8/9, pHC79, pUC19, pET, etc.), or a phage (e.g., λgt4λB, λ-Charon, λΔz1, M13, etc.), which is often used in the art.

According to an embodiment of the present invention, the nucleotide sequence encoding the Fab fragment to EGFR is cloned into pACYCDuet-1. Refer to https://www.snapgene.com/resources/plasmid_files/pet_and_duet_vectors_(novagen)/pACYCDuet-1/for information relating the pACYCDuet-1.

The recombinant vector includes a nucleotide sequence encoding a signal peptide so that the Fab fragment of the present invention is generated in E. coli, specifically, in the periplasm of E. coli.

According to an embodiment of the present invention, the signal peptide is OmpA signal peptide, LamB signal peptide, StlI signal peptide, MalE signal peptide, Lpp signal peptide, and PeIB signal peptide.

According to another embodiment of the present invention, the signal peptide is OmpA signal peptide.

The OmpA signal peptide is located at the upstream of the nucleotide sequence encoding the heavy chain variable region.

The amino acid sequence encoding the OmpA signal peptide is SEQ ID NO: 3, and the nucleotide sequence encoding the OmpA signal peptide is SEQ ID NO: 8.

In accordance with another aspect of the present invention, there is provided a host cell transformed with the recombinant vector.

Host cells in which the vector of the present invention can be stably and continuously cloned and expressed are known in the art, and thus any host cell may be used, for example, intestinal microflora and strains, including E. coli C43(DE3), E. coli JM109, E. coli BL21 (DE3), E. coli RR1, E. coli LE392, E. coli B, E. coli X 1776, E. coli W3110, strains of the genus Bacillus, such as Bacillus subtilis and Bacillus thuringiensis, Salmonella typhimurium, Serratia marcescens, and various Pseudomonas Spp.

The delivery of the vector of the present invention into the host cell may be conducted by a thermal shock method, the CaCl₂ method (Cohen, S. N. et al., Proc. Natl. Acac. Sci. USA, 9:2110-2114(1973)), the Hannahan's method (Cohen, S. N. et al., Proc. Natl, Acac. Sci. USA, 9:2110-2114(1973); and Hanahan, D., J. Mal, Biol., 166:557-580(1983)), and an electroporation method (Dower, W. J. et al., Nucleic. Acids Res.,

According to an embodiment of the present invention, the host cell of he present invention is E. coli.

According to another embodiment of the present invention, the host cell of the present invention is E. coli C43(DE3). For information relating to E. coli C43(DE3), refer to The toxicity of recombinant proteins in Escherichia coil: a comparison of overexpression in BL21(DE3), C41(DE3), and C43(DE3), published through Protein Expression and Purification 37(2004) 203-206, by Laurence Dumon-Seignovert et al.

In accordance with still another aspect of the present invention, there is provided a method for preparing a fragment antigen-binding (Fab) fragment specifically binding epidermal growth factor receptor (EGFR), the method including:

(a) culturing the host cells, which are transformed with the recombinant vector including the amino acid sequence encoding the Fab fragment to EGFR; and

(b) expressing the Fab fragment to EGFR in the host cells.

Since the method of the present invention is directed to a method for manufacturing the Fab fragment to EGFR, descriptions of overlapping contents therebetween are omitted to avoid excessive complication of the specification.

The host cells in step (a) of the present invention may be cultured by various culturing methods known in the art.

According to an embodiment of the present invention, the host cells are cultured in at least one culture medium selected from the group consisting of super broth (SB), fastidious broth (FB), lysogeny broth (LB), terrific broth (TB), super optimal broth with catabolic repressor (SOC), and super optimal broth (SOB).

According to another embodiment of the present invention, the host cells are cultured in at least one culture medium selected from the group consisting of super broth (SB), fastidious broth (FB), and lysogeny broth (LB).

According to a particular embodiment of the present invention, the host cells are cultured in super broth (SB).

In accordance with another aspect of the present invention, there is provided a pharmaceutical composition for preventing or treating cancer, including: (a) a pharmaceutically effective amount of the fragment antigen-binding (Fab) fragment specifically binding to epidermal growth factor receptor (EGFR); and (b) a pharmaceutically acceptable carrier.

According to an embodiment of the present invention, the cancer is breast cancer, large intestine cancer, lung cancer, stomach cancer, liver cancer, blood cancer, bone cancer, pancreatic cancer, skin cancer, brain cancer, cervical cancer, nasopharyngeal cancer, laryngeal cancer, colorectal cancer, ovarian cancer, rectal cancer, large intestine cancer, vaginal cancer, small intestine cancer, endocrine cancer, thyroid cancer, parathyroid cancer, ureter cancer, urinary tract cancer, prostate cancer, bronchial cancer, bladder cancer, kidney cancer, or bone marrow cancer.

According to another embodiment of the present invention, the cancer is head and neck cancer.

As used herein, the term “pharmaceutically effective amount” refers to an amount sufficient to attain efficacy or activity of the foregoing Fab fragment to EGFR compound.

In cases where the Fab fragment of the present invention is prepared as a pharmaceutical composition, the pharmaceutical composition of the present invention contains a pharmaceutically acceptable carrier. The pharmaceutically acceptable carrier contained in the pharmaceutical composition of the present invention is conventionally used for the formulation, and examples thereof may include, but are not limited to, lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate, and mineral oil. The pharmaceutical composition of the present invention may further contain, in addition to the above components, a lubricant, a wetting agent, a sweetening agent, a flavoring agent, an emulsifier, a suspending agent, a preservative, and the like. Suitable pharmaceutically acceptable carriers and agents are described in detail in Remington's Pharmaceutical Sciences (19th ed., 1995).

The pharmaceutical composition of the present invention may be administered orally or parenterally, and preferably, the parenteral administration manner is employed.

A suitable dose of the pharmaceutical composition of the present invention may vary depending on various factors, such as a method of formulation, manner of administration, the age, body weight, gender, and morbidity of the patient, diet, time of administration, excretion rate, and response sensitivity. A general dose of the pharmaceutical composition of the present invention is within the range of 0.001 μg/kg-1000 mg/kg in adults.

The pharmaceutical composition of the present invention may be formulated into a unit or multiple dosages form using a pharmaceutically acceptable carrier and/or excipient according to the method easily conducted by a person having ordinary skill in the art to which the present invention pertains. Here, the dosage form may be a solution in an oily or aqueous medium, a suspension, a syrup, or an emulsion, an extract, a powder, a granule, a tablet, or a capsule, and may further include a dispersant or a stabilizer.

### Advantageous Effects

Features and advantages of the present invention are summarized as follows:

(a) The present invention provides a fragment antigen-binding (Fab) fragment specifically binding to epidermal growth factor receptor (EGFR), an expression construct for preparing the Fab fragment, a method for preparing the Fab fragment, and a pharmaceutical composition containing the Fab fragment.

(b) The Fab fragment to EGFR of the present invention is smaller than the antibody, and thus can favorably permeate into tissues or tumors and can be prepared in bacteria, resulting in low production costs.

(c) The Fab fragment to EGFR of the present invention has an increased in vivo half-life through pegylation.

### BRIEF DESCRIPTION OF THE DRAWINGS

FIG. 1 illustrates Fm301 construct and pACYCDuet-1 vector map.

FIG. 2 illustrates confirmation results of Fm301 cloning through polymerase cloning reaction (PCR).

FIG. 3 illustrates Fm302 construct and pACYCDuet-1 vector map.

FIG. 4 illustrates confirmation results of Fm302 cloning through PCR.

FIG. 5 illustrates Fm306 construct and pACYCDuet-1 vector map.

FIG. 6 illustrates confirmation results of Fm306 cloning through PCR.

FIGS. 7a to 7c illustrate confirmation results of Fm301 and Fm302 proteins expressed in E. coli through SDS-PAGE, and results of binding the proteins with anti-Fab antibody.

FIG. 8 illustrates analysis results of homogeneity of Fm301 and Fm302 after purification.

FIG. 9 illustrates confirmation results of Fm306 protein expressed in E. coli through SDS-PAGE.

FIGS. 10a and 10b illustrate antibody purification yields for different media (Lysogeny broth; LB, Fantidious broth; FB, and Super broth; SB).

FIG. 11 illustrates electrophoresis results of Fm302 expressed in E. coli, using non-reducing dye.

FIGS. 12a and 12b illustrate ion exchange chromatography results of Fm302.

FIG. 13 illustrates size exclusion chromatography results of Fm302.

FIG. 14 illustrates confirmation results of disulfide bonds formed by adding Cys-Asp-Lys and Glu-Cys to C-terminals of C_{H1} and C_{L} of Fm302, respectively.

FIG. 15 illustrates confirmation results of Fm306 purification through SDS-PAGE.

FIG. 16 illustrates confirmation results of preparation and yields of Fm306-PEG(20K) and Fm306-PEG(30K) conjugates.

FIG. 17 illustrates structures of Fab' constructs. Fm301, Fm302, and Fm306 all have four intra-chain disulfide bonds, equally, and each of Fm302 and Fm306 has an intra-chain disulfide bond at the C-terminal thereof. Fm306 has an amino acid at the C-terminal of the heavy chain, the amino acid being added for pegylation,

FIGS. 18a to 18d illustrate confirmation results of the pegylation of Fm306-PEG through SDS-PAGE, PEG staining, and western blot. FIG. 18a illustrates SDS-PAGE coomassie blue staining results of Fm301, Fm302, and Fm306 samples and respective construct samples subjected to an attempt to react with PEG. FIG. 18b illustrates PEG staining results of the same samples as in FIG. 18a. FIG. 18c illustrates western blot results using an anti-Fab antibody, of Fm301 Fm302, and Fm306 samples and respective construct samples subjected to an attempt to react with PEG. FIG. 18d illustrates western blot results using an anti-Fab antibody, of Fm301, Fm302, and Fm306 samples and respective construct samples subjected to an attempt to react with PEG.

FIGS. 19a to 19d illustrate confirmation results of the pegylation of Fm306-PEG after the removal of residual PEG through SDS-PAGE, PEG staining, and western blot. FIG. 19a illustrates SDS-PAGE coomassie blue staining results of Fm301, Fm302, and Fm306 construct samples after the removal of residual PEG. FIG. 19b illustrates PEG staining results of the same samples as in FIG. 19a. FIG. 19c illustrates western blot results using an anti-Fab antibody, of Fm301, Fm302, and Fm306 construct samples after the removal of residual PEG. FIG. 19d illustrates western blot results using an anti-Fab antibody, of Fm301, Fm302, and Fm306 construct samples after the removal of residual PEG.

FIGS. 20a to 20d illustrate kinetic values of cetuximab, cetuximab-Fab, Fm302, and Fm306FEG, respectively.

FIG. 21 illustrates sEGFR-binding affinity of Fm302 and Fm306PEG.

FIG. 22 illustrates EGFR phosphorylation degrees of cetuximab, cetuximab-Fab, Fm302, and Fm306FEG.

FIG. 23 illustrates tumor tissue growth in the head and neck cancer disease animal model by cetuximab, cetuximab-Fab, Fm302, and Fm306FEG in the head and neck cancer disease animal model.

FIG. 24 illustrates tumor tissue sizes at the time of autopsy of the head and neck cancer disease animal model, by cetuximab, cetuximab-Fab, Fm302, and Fm306FEG.

### MODE FOR CARRYING OUT THE INVENTION

Hereinafter, the present invention will be described in detail with reference to examples. These examples are only for illustrating the present invention more specifically, and it will be apparent to those skilled in the art that the scope of the present invention is not limited by these examples.

### EXAMPLE 1

### Preparation of Fab Construct

Intra-chain disulfide bonds, which, respectively, exist in the light chain variable region (V_{L}) and the heavy chain variable region (V_{H}), among domains constituting an antibody, stabilize structures of the respective domains. These intra-chain disulfide bonds are known to play an important role in the interaction between an antibody (Anti-EGFR) and an antigen (EGFR(HER1)) [(Yang. et al,, PNAS. 104(26):10813-10817(2007): and Liu, H. and May, k., MAbs, 4:17-23(2012)]. Also, it is known that two cysteines existing in the hinge domain form an intra-chain disulfide bond between respective domains, which performs a structurally important role when the antibody configures a dimer [K. Zangger. et al., Biochem, 359:353-360(2001), Levy, R. et al., J. Immunol. Methods., 394:10-21(2013)]. However, the anti-EGFR antibody fragment, developed in the present invention, has no a hinge domain, resulting in a deletion of the hinge domain, thereby forming Fab′, and thus monovalent antibody fragments can be produced. Meanwhile, it is known that, when V_{H}+C_{H1} and V_{L}+C_{L} domains are respectively expressed and the folding thereof is induced in the periplasm of E. coli, the domains can be folded in a stable structure (S. Ewert. et al,, J. Mol. Biol. 325:531-553(2003)). In order to express each domain of the antibody fragment and send each domain into the periplasm of E. coli, the OmpA signal peptide was introduced at the front of each domain.

Fab′ Construct [V_{H}−C_{H1}(‘CDK’ Deletion), V_{L}−C_{L}: Fm301]

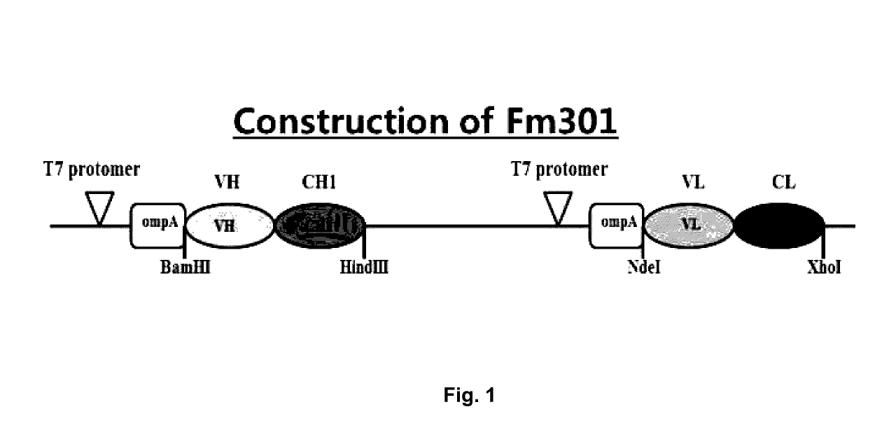

Fab′, which is monovalent Fab, as an antibody fragment platform, was prepared, and then called Fm. In order to clone V_{H}+C_{H1} and V_{L}+C_{L} domains of Fab, the synthesis of DNA sequences (tables 4 and 5) for amino acid sequences (tables 2 and 3) of signal peptide (OmpA)+V_{H}+C_{H1} and signal peptide (OmpA)+V_{L}+C_{L} were requested at Cosmo Genentech Inc., on the basis of amino acid sequences (table 1) of cetuximab. In table 1, underlined parts in the sequences represent variable regions. PCR primers (table 6) were prepared at sites corresponding to V_{H}+C_{H1} and V_{L}+C_{L} domains by using the DNA nucleotide sequences, and genes were cloned to express signal peptide (OmpA)+V_{H}+C_{H1} and signal peptide (OmpA)+V_{L}+C_{L}, respectively, and then cloned into pACYCDuet-1 vector (Novagen), which is a co-expression vector of E. coli, through treatment with restriction enzymes (FIG. 1).

TABLE 1

| | | SEQ |
|---|---|---|
| | | ID |
| — | Sequence | NO |
| Anti- | QVQLKQSGPGLVQPSQSLSITCTVSGFSLTNYGVHWVR | 1 |
| EGFR | QSPGKGLEWLGVIWSGGNTDYNTPFTSRLSINKDNSKS | |
| heavy | QVFFKMNSLQSNDTAIYYCARALTYYDYEFAYWGQGTL | |
| chain | VTVSAASTKGPSVFPLAPSSKSTSGGTAALGCLVKDYF | |
| | PEPVTVSWNSGALTSGVHTFPAVLQSSGLYSLSSVVTV | |
| | PSSSLGTQTYICNVNHKPSNTKVDKRVEPKSPKSCDKT | |
| | HTCPPCPAPELLGGPSVFLFPPKPKDTLMISRTPEVTC | |
| | VVVDVSHEDPEVKFNWYVDGVEVHNAKTKPREEQYNST | |
| | YRVVSVLTVLHQDWLNGKEYKCWSNKALPAPIEKTISK | |
| | AKGQPREPQVYTLPPSRDELTKNQVSLTCLVKGFYPSD | |
| | IAVEWESNGQPENNYKTTPPVLDSDGSFFLYSKLTVDK | |
| | SRWQQGNVFSCSVMHEALHNHYTQKSLSLSPGK | |
| Anti- | DILLTQSPVILSVSPGERVSFSCRASQSIGTNIHWYQQ | 2 |
| EGFR | RTNGSPRLLIKYASESISGIPSRFSGSGSGTDFTLSIN | |
| light | SVESEDIADYYCQQNNNWPTTFGAGTKLELKRTVAAPS | |
| chain | VFIFPPSDEQLKSGTASVVCLLNNFYPREAKVQWKVDN | |
| | ALQSGNSQESVTEQDSKDSTYSLSSTLTLSKADYEKHK | |
| | VYACEVTHQGLSSPVTKSFNRGA | |

TABLE 2

| | | SEQ |
|---|---|---|
| | | ID |
| — | Sequence | NO |
| OmpA | MKKTAAIAVALAGFATVAQA | 3 |
| signal | | |
| peptide | | |
| V_{H} | QVQLKQSGPGLVQPSQSLSITCTVSGFSLTNYGVHW | 4 |
| | VRQSPGKGLEWLGVIWSGGNTDYNTPFTSRLSINKD | |
| | NSKSQVFFKMNSLQSNDTAIYYCARALTYYDYEFAY | |
| | WGQGTLVTVSA | |
| C_{H1} | ASTKGPSVFPLAPSSKSTSGGTAALGCLVKDYFPEPV | |
| | TVSWNSGALTSGVHTFPAVLQSSGLYSLSSVVTVPS | 5 |
| | SSLGTQTYICNVNHKPSNTKVDKRVEPKS | |

TABLE 3

| | | SEQ |
|---|---|---|
| | | ID |
| — | Sequence | NO |
| OmpA | MKKTAIAIAVALAGFATVAQA | 3 |
| signal | | |
| peptide | | |
| V_{L} | DILLTQSPVILSVSPGERVSFSCRASQSIGTNIHWYQQ | 6 |
| | RTNGSPRLLIKYASESISGIPSRFSGSGSGTDFTLSINS | |
| | VESEDIADYYCQQNNNWPTTFGAGTKLELK | |
| C_{L} | RTVAAPSVFIFPPSDEQLKSGTASVVCLLNNFYPREA | 7 |
| | KVQWKVDNALQSGNSQESVTEQDSKDSTYSLSSTLT | |
| | LSKADYEKHKVYACEVTHQGLSSPVTKSFNRGA | |

TABLE 4

| | | SEQ |
|---|---|---|
| | | ID |
| — | Sequence | NO |
| OmpA | ATGAAAAAGACAGCTATCGCGATTGCAGTGGCACTG | 8 |
| signal | GCTGGTTTCGCTACCGTAGCGCAGGCC | |
| peptide | | |
| V_{H} | CAAGTCCAACTGAAACAATCGGGTCCGGGTCTGGTC | 9 |
| | CAACCGTCCCAATCACTGAGCATCACCTGTACCGTG | |
| | TCGGGCTTCTCGCTGACCAATTATGGTGTGCATTGG | |
| | GTTCGTCAGAGTCCGGGCAAAGGTCTGGAATGGCT | |
| | GGGCGTTATTTGGTCCGGCGGTAATACCGATTACAA | |
| | CACCCCGTTTACGAGTCGCCTGTCCATCAATAAAGA | |
| | CAACTCGAAAAGCCAGGTGTTTTTCAAAATGAATTCA | |
| | CTGCAATCGAACGATACCGCGATTTATTACTGCGCA | |
| | CGTGCTCTGACGTATTACGACTATGAATTTGCCTACT | |
| | GGGGCCAGGGTACCCTGGTGACGGTTAGCGCG | |
| C_{H1} | GCCTCTACCAAAGGTCCGAGCGTTTTCCCGCTGGCA | 10 |
| | CCGAGCTCTAAATCTACCAGTGGCGGTACGGCAGCT | |
| | CTGGGCTGTCTGGTGAAAGATTATTTTCCGGAACCG | |
| | GTCACCGTGAGTTGGAATTCCGGTGCACTGACCAGT | |
| | GGCGTCCACACGTTCCCGGCTGTGCTGCAGAGTTC | |
| | CGGTCTGTATAGCCTGTCATCGGTGGTTACCGTTCC | |
| | GAGCTCTAGTCTGGGCACCCAAACGTACATTTGCAA | |
| | TGTCAACCATAAACCGAGCAACACGAAAGTTGATAAA | |
| | CGTGTCGAACCGAAATCA | |

TABLE 5

| | | SEQ |
|---|---|---|
| | | ID |
| — | Sequence | NO |
| OmpA | ATGAAAAAGACAGCTATCGCGATTGCAGTGGCACTG | 8 |
| signal | GCTGGTTTCGCTACCGTAGCGCAGGCC | |
| peptide | | |
| V_{L} | GATATTCTGCTGACCCAGAGCCCGGTGATCCTGAGT | 11 |
| | GTTTCCCCGGGCGAACGTGTGTCATTTTCGTGTCGC | |
| | GCGAGCCAGTCTATTGGTACCAATATCCACTGGTAT | |
| | CAGCAACGTACGAACGGCTCTCCGCGCCTGCTGATT | |
| | AAATACGCCAGTGAATCCATTTCAGGCATCCCGAGC | |
| | CGCTTTTCGGGCAGCGGTTCTGGCACCGATTTCACG | |
| | CTGAGTATTAACTCCGTGGAATCAGAAGATATCGCA | |
| | GACTATTACTGCCAGCAAAACAATAACTGGCCGACC | |
| | ACGTTTGGTGCTGGCACCAAACTGGAACTGAAA | |
| C_{L} | CGTACGGTGGCGGCCCCGAGTGTTTTTATCTTCCCG | 12 |
| | CCGTCCGATGAACAGCTGAAATCGGGTACCGCCAG | |
| | CGTTGTCTGTCTGCTGAATAACTTCTATCCGCGCGA | |
| | AGCAAAAGTCCAGTGGAAAGTGGACAATGCTCTGCA | |
| | GTCGGGCAACAGCCAAGAAAGCGTGACCGAACAAG | |
| | ATAGTAAAGACTCCACGTACTCACTGTCCTCAACCCT | |
| | GACGCTGAGCAAAGCGGATTATGAAAAACACAAAGT | |
| | GTACGCCTGCGAAGTTACCCATCAAGGTCTGAGTAG | |
| | CCCGGTTACGAAATCATTCAATCGTGGTGCC | |

TABLE 6

| | | SEQ |
|---|---|---|
| | | ID |
| Primer | Sequence (5′ → 3′) | NO |
| Vₕ domain | CGCCCATGGCCAAAAAGACAACAGCTATCGC | 13 |
| forward primer | GATTGC | |
| Ch₁ domain | ATGCGGCCGCAAGCTTCTATGATTTCGGTTCG | 14 |
| reverse primer | ACACG | |
| Vₗ domain | AAGGAGATATACATATGAAAAAGACAGCTATC | 15 |
| forward primer | GCGATTGCAGTGGCACTG GCTGGTT | |
| Cₗ domain | CTTTACCAGACTCGAGCTAGGCACCACGATTG | 16 |
| reverse primer | AATGA | |

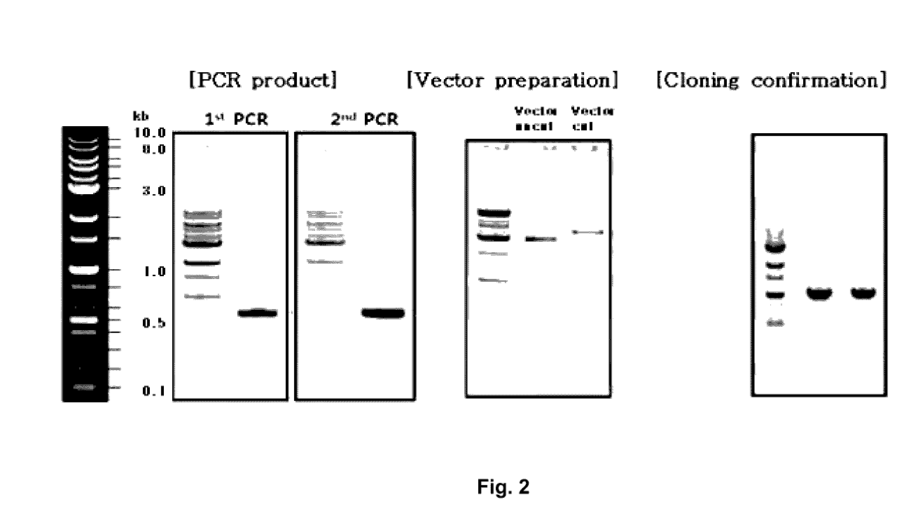

The Fm-301 construct was PCR-amplified, and the cloning results thereof were confirmed (FIG. 2).

Fab′ Construct (V_{H}−C_{H1}, V_{L}−C_{L}(+EC): Fm302)

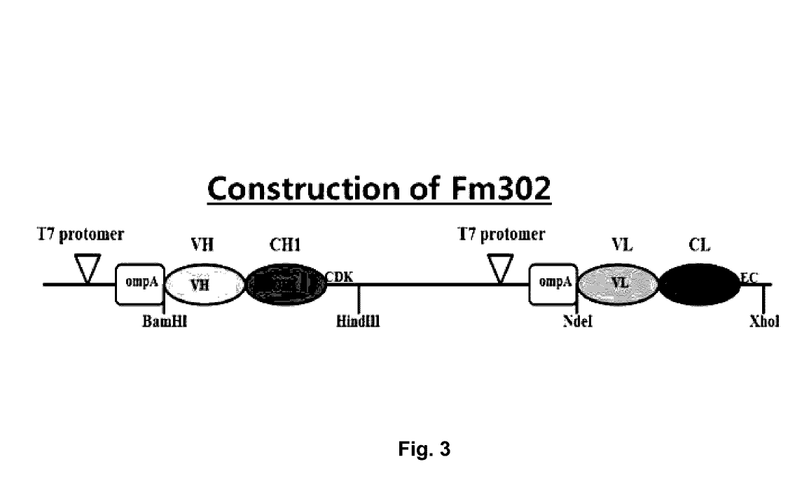

In order to clone constructs (tables 7 and 8) in which amino acid sequences “CDK” and “EC” are respectively inserted into the C-terminals of C_{H1} and C_{L} of the Fm301 construct, the synthesis of DNA nucleotide sequences (tables 9 and 10), which could be expressed in E. coli through codon conversion of the clone constructs, were requested at Cosmo Genentech Inc. The “CDK” and “EC” were added to induce a disulfide bond for allowing the light chain and the heavy chain to form a heterodimer. PCR primers (table 11) were prepared at sites corresponding to V_{H}+C_{H1}(+CDK) and V_{L}+C_{L}(+EC) domains by using the DNA nucleotide sequences, and genes were cloned to express signal peptide (OmpA)+V_{H}+C_{H1}(+CDK) and signal peptide (OmpA)+V_{L}+C_{L}(+EC) domains, respectively, and then cloned into pACYCDuet-1 vector (Novagen), which is a co-expression vector of E. coli, through treatment with restriction enzymes (FIG. 3).

TABLE 7

| | | SEQ |
|---|---|---|
| | | ID |
| — | Sequence | NO |
| OmpA | MKKTAIAIAVALAGFATVAQA | 3 |
| signal | | |
| peptide | | |
| V_{H} | QVQLKQSGPGLVQPSOSLSITCTVSGFSLTNYGVHW | 4 |
| | VRQSPGKGLEWLGVIWSGGNTDYNTPFTSRLSINKD | |
| | NSKSQVFFKMNSLQSNDTAIYYCARALTYYDYEFAY | |
| | WGQGTLVTVSA | |
| C_{H1} + CDK | ASTKGPSVFPLAPSSKSTSGGTAALGCLVKDYFPEPV | 17 |
| | TVSWNSGALTSGVHTFPAVLQSSGLYSLSSVVTVPS | |
| | SSLGTQTYICNVNHKPSNTKVDKRVEPKSCDK | |

TABLE 8

| | | SEQ |
|---|---|---|
| | | ID |
| — | Sequence | NO |
| OmpA | MKKTAIAIAVALAGFATVAQA | 3 |
| signal | | |
| peptide | | |
| V_{L} | DILLTQSPVILSVSPGERVSFSCRASQSIGTNIHWYQQ | 6 |
| | RTNGSPRLLIKYASESISGIPSRFSGSGSGTDFTLSINS | |
| | VESEDIADYYCQQNNNWPTTFGAGTKLELK | |
| C_{L} + EC | RTVAAPSVFIFPPSDEQLKSGTASWCLLNNFYPREA | 18 |
| | KVQWKVDNALQSGNSQESVTEQDSKDSTYSLSSTLT | |
| | LSKADYEKHKVYACEVTHQGLSSPVTKSFNRGAEC | |

TABLE 9

| | | SEQ |
|---|---|---|
| | | ID |
| — | Sequence | NO |
| OmpA | ATGAAAAAGACAGCTATCGCGATTGCAGTGGCACT | 8 |
| signal | GGCTGGTTTCGCTACCGTAGCGCAGGCC | |
| peptide | | |
| V_{H} | CAAGTCCAACTGAAACAATCGGGTCCGGGTCTGGT | 9 |
| | CCAACCGTCCCAATCACTGAGCATCACCTGTACCG | |
| | TGTCGGGCTTCTCGCTGACCAATTATGGTGTGCAT | |
| | TGGGTTCGTCAGAGTCCGGGCAAAGGTCTGGAAT | |
| | GGCTGGGCGTTATTTGGTCCGGCGGTAATACCGAT | |
| | TACAACACCCCGTTTACGAGTCGCCTGTCCATCAA | |
| | TAAAGACAACTCGAAAAGCCAGGTGTTTTTCAAAAT | |
| | GAATTCACTGCAATCGAACGATACCGCGATTTATTA | |
| | CTGCGCACGTGCTCTGACGTATTACGACTATGAAT | |
| | TTGCCTACTGGGGCCAGGGTACCCTGGTGACGGT | |
| | TAGCGCG | |
| C_{H1} + CDK | GCCTCTACCAAAGGTCCGAGCGTTTTCCCGCTGGC | 19 |
| | ACCGAGCTCTAAATCTACCAGTGGCGGTACGGCAG | |
| | CTCTGGGCTGTCTGGTGAAAGATTATTTTCCGGAA | |
| | CCGGTCACCGTGAGTTGGAATTCCGGTGCACTGAC | |
| | CAGTGGCGTCCACACGTTCCCGGCTGTGCTGCAG | |
| | AGTTCCGGTCTGTATAGCCTGTCATCGGTGGTTAC | |
| | CGTTCCGAGCTCTAGTCTGGGCACCCAAACGTACA | |
| | TTTGCAATGTCAACCATAAACCGAGCAACACGAAA | |
| | GTTGATAAACGTGTCGAACCGAAATCATGCGATAA | |
| | A | |

TABLE 10

| | | SEQ |
|---|---|---|
| | | ID |
| — | Sequence | NO |
| OmpA | ATGAAAAAGACAGCTATCGCGATTGCAGTGGCACT | 8 |
| signal | GGCTGGTTTCGCTACCGTAGCGCAGGCC | |
| peptide | | |
| V_{L} | GATATTCTGCTGACCCAGAGCCCGGTGATCCTGAG | 11 |
| | TGTTTCCCCGGGCGAACGTGTGTCATTTTCGTGTC | |
| | GCGCGAGCCAGTCTATTGGTACCAATATCCACTGG | |
| | TATCAGCAACGTACGAACGGCTCTCCGCGCCTGCT | |
| | GATTAAATACGCCAGTGAATCCATTTCAGGCATCCC | |
| | GAGCCGCTTTTCGGGCAGCGGTTCTGGCACCGATT | |
| | TCACGCTGAGTATTAACTCCGTGGAATCAGAAGAT | |
| | ATCGCAGACTATTACTGCCAGCAAAACAATAACTG | |
| | GCCGACCACGTTTGGTGCTGGCACCAAACTGGAAC | |
| | TGAAA | |
| C_{L} + EC | CGTACGGTGGCGGCCCCGAGTGTTTTTATCTTCCC | 20 |
| | GCCGTCCGATGAACAGCTGAAATCGGGTACCGCC | |
| | AGCGTTGTCTGTCTGCTGAATAACTTCTATCCGCG | |
| | CGAAGCAAAAGTCCAGTGGAAAGTGGACAATGCTC | |
| | TGCAGTCGGGCAACAGCCAAGAAAGCGTGACCGA | |
| | ACAAGATAGTAAAGACTCCACGTACTCACTGTCCTC | |
| | AACCCTGACGCTGAGCAAAGCGGATTATGAAAAAC | |
| | ACAAAGTGTACGCCTGCGAAGTTACCCATCAAGGT | |
| | CTGAGTAGCCCGGTTACGAAATCATTCAATCGTGG | |
| | TGCCGAATGC | |

TABLE 11

| | | SEQ |
|---|---|---|
| | | ID |
| Primer | Sequence(5′ → 3′) | NO |
| Vₕ domain | CGCCCATGGCCAAAAAGACAACAGCTATCG | 13 |
| forward | CGATTGC | |
| primer | | |
| Cₕ₁ + cdk | ATGCGGCCGCAAGCTTCTATTTATCGCATGA | 21 |
| domain | TTTCGGTTCGACACG | |
| reverse | | |
| primer | | |
| Vₗ domain | AAGGAGATATACATATGAAAAAGACAGCTAT | 15 |
| forward | CGCGATTGCAGTGGCACTG GCTGGTT | |
| primer | | |
| Cₗ + ec domain | CTTTACCAGACTCGAGCTAGCATTCGGCAC | 22 |
| reverse primer | CACGATTGAATGA | |

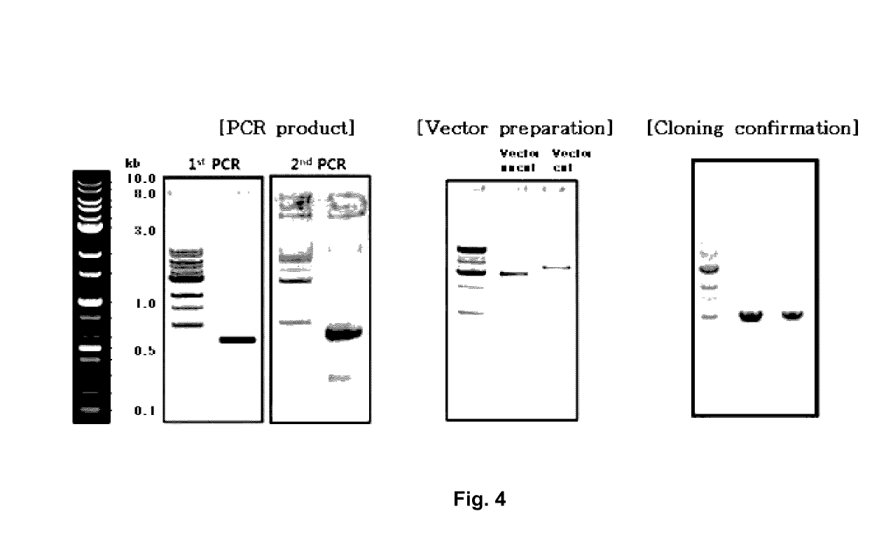

The Fm-302 construct was PCR-amplified, and the cloning results thereof were confirmed (FIG. 4)

Fab′ Construct (V_{H}+C_{H1}+THTCAA, V_{L}+C_{L}+EC: Fm306)

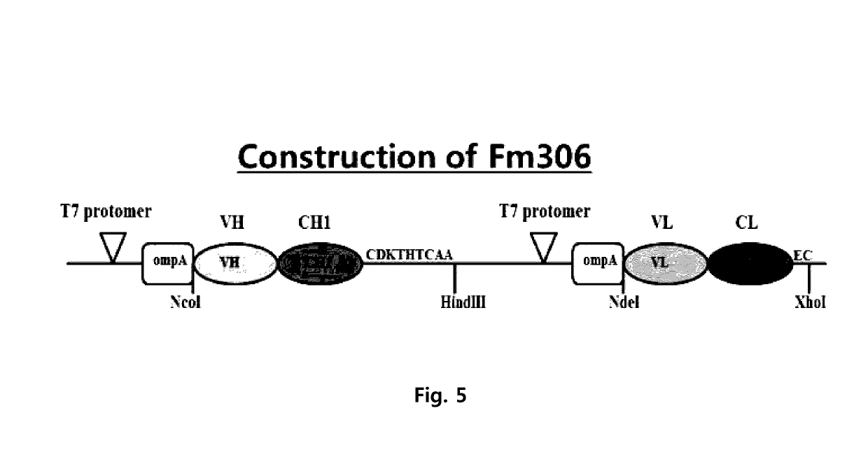

For a site specific pegylation reaction of the Fm302 gene prepared above, DNA nucleotide sequences (tables 14 and 15) for the construct (table 12) which has THTCAA, amino acids at the hinge site, inserted into the C-terminal of the C_{H1} domain and could be expressed in E. coli through codon conversion, were requested to be synthesized by Cosmo Genentech Inc. PCR primers (table 16) were prepared at the sites corresponding to V_{H}+C_{H1}+THTCAA and V_{L}+C_{L}+EC by using the DNA nucleotide sequences, followed by E. coli expression PCR, and the genes were cloned into pACYCDuet-1 vector, which is a co-expression vector of E. coli, through treatment with restriction enzymes (FIG. 5),

TABLE 12

| | | SEQ |
|---|---|---|
| | | ID |
| — | Sequence | NO |
| OmpA signal | MKKTAIAIAVALAGFATVAQA | 3 |
| peptide | | |
| V_{H} | QVQLKQSGPGLVQPSQSLSITCTVSGFSLTNYGVHWV | 4 |
| | RQSPGKGLEWLGVIWSGGNTDYNTPFTSRLSINKDNS | |
| | KSQVFFKMNSLQSNDTAIYYCARALTYYDYEFAYWGQ | |
| | GTLVTVSA | |
| C_{H1} + | ASTKGPSVFPLAPSSKSTSGGTAALGCLVKDYFPEPV | 23 |
| CDK + | TVSWNSGALTSGVHTFPAVLQSSGLYSLSSVVTVPSS | |
| THTCAA | SLGTQTYICNVNHKPSNTKVDKRVEPKSCDKTHTCAA | |

TABLE 13

| | | SEQ |
|---|---|---|
| | | ID |
| — | Sequence | NO |
| OmpA | MKKTAIAIAVALAGFATVAQA | 3 |
| signal | | |
| peptide | | |
| V_{L} | DILLTQSPVILSVSPGERVSFSCRASQSIGTNIHWYQQ | 6 |
| | RTNGSPRLLIKYASESISGIPSRFSGSGSGTDFTLSINS | |
| | VESEDIADYYCQQNNNWPTTFGAGTKLELK | |
| C_{L} + EC | RTVAAPSVFIFPPSDEQLKSGTASVVCLLNNFYPREAK | 18 |
| | VQWKVDNALQSGNSQESVTEQDSKDSTYSLSSTLTLS | |
| | KADYEKHKVYACEVTHQGLSSPVTKSFNRGAEC | |

TABLE 14

| | | SEQ |
|---|---|---|
| | | ID |
| — | Sequence | NO |
| OmpA | ATGAAAAAGACAGCTATCGCGATTGCAGTGGCACTG | 8 |
| signal | GCTGGTTTCGCTACCGTAGCGCAGGCC | |
| peptide | | |
| V_{H} | CAAGTCCAACTGAAACAATCGGGTCCGGGTCTGGTC | 9 |
| | CAACCGTCCCAATCACTGAGCATCACCTGTACCGTG | |
| | TCGGGCTTCTCGCTGACCAATTATGGTGTGCATTGG | |
| | GTTCGTCAGAGTCCGGGCAAAGGTCTGGAATGGCT | |
| | GGGCGTTATTTGGTCCGGCGGTAATACCGATTACAA | |
| | CACCCCGTTTACGAGTCGCCTGTCCATCAATAAAGA | |
| | CAACTCGAAAAGCCAGGTGTTTTTCAAAATGAATTCA | |
| | CTGCAATCGAACGATACCGCGATTTATTACTGCGCA | |
| | CGTGCTCTGACGTATTACGACTATGAATTTGCCTACT | |
| | GGGGCCAGGGTACCCTGGTGACGGTTAGCGCG | |
| C_{H1} + | GCCTCTACCAAAGGTCCGAGCGTTTTCCCGCTGGCA | 24 |
| CDK + | CCGAGCTCTAAATCTACCAGTGGCGGTACGGCAGCT | |
| THTCAA | CTGGGCTGTCTGGTGAAAGATTATTTTCCGGAACCG | |
| | GTCACCGTGAGTTGGAATTCCGGTGCACTGACCAGT | |
| | GGCGTCCACACGTTCCCGGCTGTGCTGCAGAGTTC | |
| | CGGTCTGTATAGCCTGTCATCGGTGGTTACCGTTCC | |
| | GAGCTCTAGTCTGGGCACCCAAACGTACATTTGCAA | |
| | TGTCAACCATAAACCGAGCAACACGAAAGTTGATAA | |
| | ACGTGTCGAACCGAAATCATGCGATAAAACCCATAC | |
| | CTGCGCGGCG | |

TABLE 15

| | | SEQ |
|---|---|---|
| | | ID |
| — | Sequence | NO |
| OmpA | ATGAAAAAGACAGCTATCGCGATTGCAGTGGCACTG | 8 |
| signal | GCTGGTTTCGCTACCGTAGCGCAGGCC | |
| peptide | | |
| VL | GATATTCTGCTGACCCAGAGCCCGGTGATCCTGAGT | 11 |
| | GTTTCCCCGGGCGAACGTGTGTCATTTTCGTGTCGC | |
| | GCGAGCCAGTCTATTGGTACCAATATCCACTGGTAT | |
| | CAGCAACGTACGAACGGCTCTCCGCGCCTGCTGATT | |
| | AAATACGCCAGTGAATCCATTTCAGGCATCCCGAGC | |
| | CGCTTTTCGGGCAGCGGTTCTGGCACCGATTTCACG | |
| | CTGAGTATTAACTCCGTGGAATCAGAAGATATCGCA | |
| | GACTATTACTGCCAGCAAAACAATAACTGGCCGACC | |
| | ACGTTTGGTGCTGGCACCAAACTGGAACTGAAA | |
| C_{L} + EC | CGTACGGTGGCGGCCCCGAGTGTTTTTATCTTCCCG | 20 |
| | CCGTCCGATGAACAGCTGAAATCGGGTACCGCCAG | |
| | CGTTGTCTGTCTGCTGAATAACTTCTATCCGCGCGA | |
| | AGCAAAAGTCCAGTGGAAAGTGGACAATGCTCTGCA | |
| | GTCGGGCAACAGCCAAGAAAGCGTGACCGAACAAG | |
| | ATAGTAAAGACTCCACGTACTCACTGTCCTCAACCCT | |
| | GACGCTGAGCAAAGCGGATTATGAAAAACACAAAGT | |
| | GTACGCCTGCGAAGTTACCCATCAAGGTCTGAGTAG | |
| | CCCGGTTACGAAATCATTCAATCGTGGTGCCGAATG | |
| | C | |

TABLE 16

| | | SEQ |
|---|---|---|
| | | ID |
| — | Sequence (5′ → 3′) | NO |
| V_{H} domain | CGCCCATGGCCAAAAAGACAACAGCTATCGCG | 13 |
| forward | ATTGC | |
| primer | | |
| C_{H1} + CDK + | CGCAAGCTTCTACGCCGCGCAGGTATGGGTTTT | 25 |
| THAC AA | ATCGCATGA TTTCGGTTC | |
| domain | | |
| reverse | | |
| primer | | |

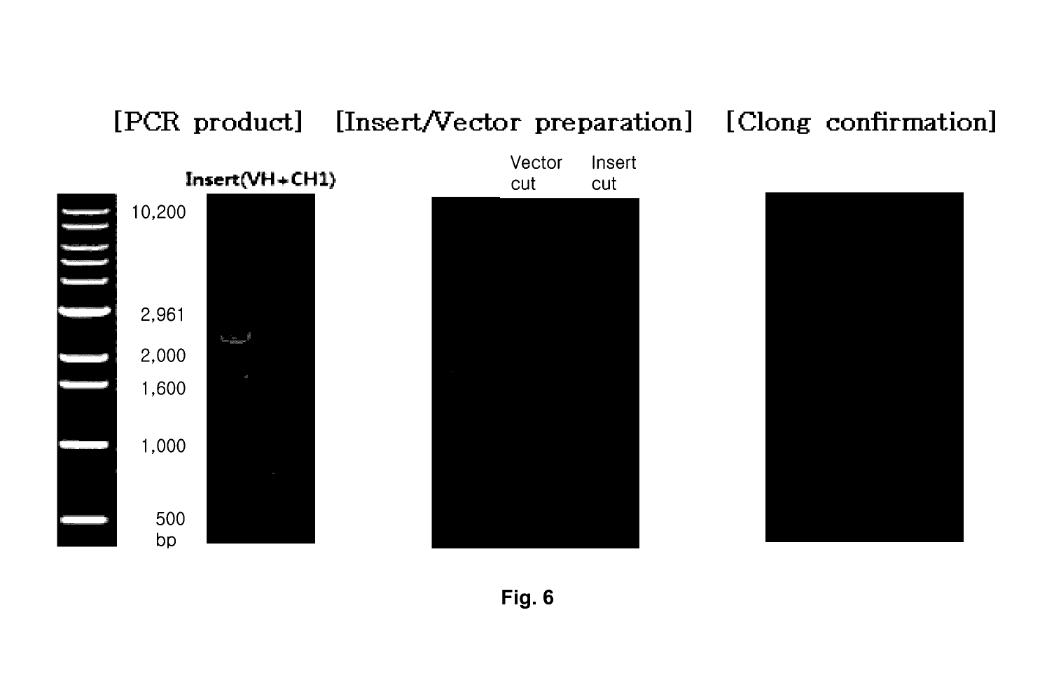

The Fm-306 construct was PCR-amplified, and the cloning results thereof were confirmed (FIG. 6).

### EXAMPLE 2

### Confirmation on Expression of Fm301 and Fm302 Constructs in E. coli

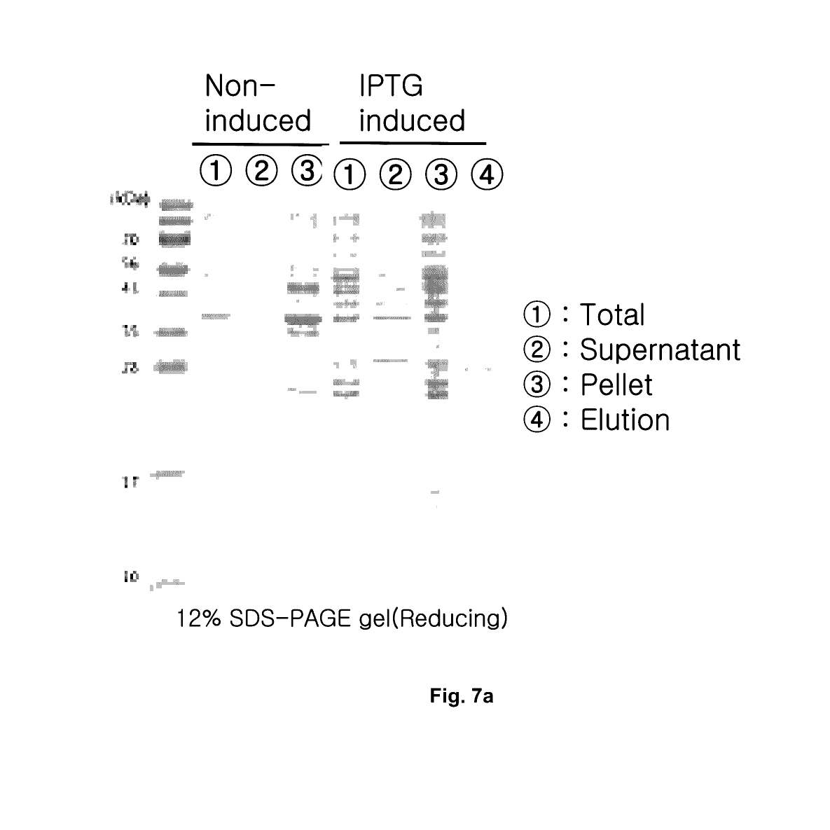

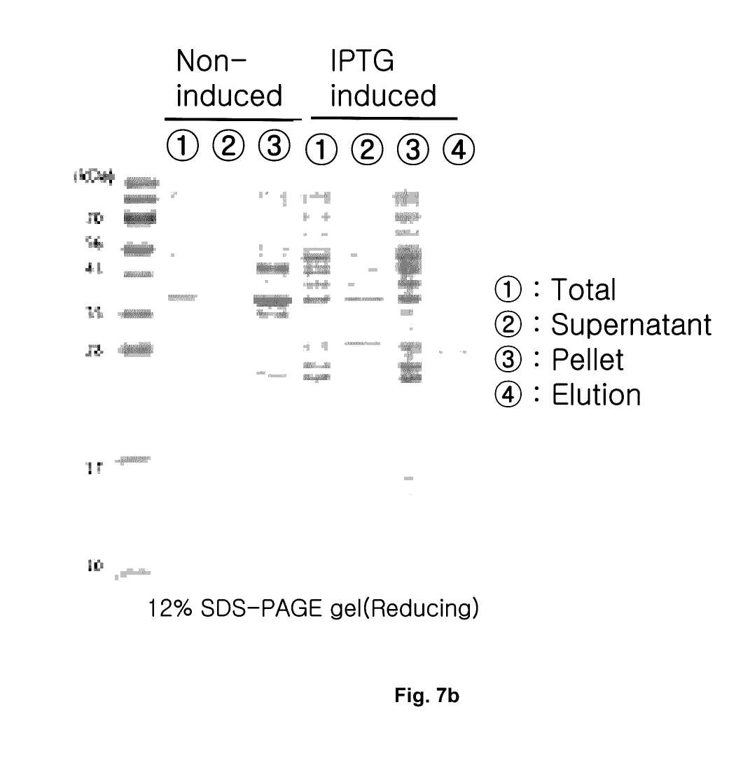

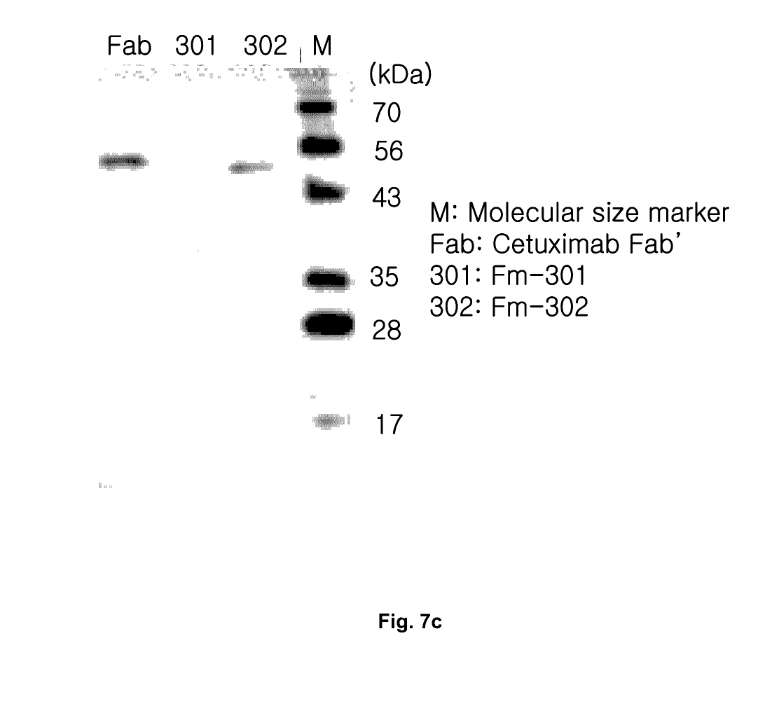

For the confirmation of genetic expression of Fm301 and Fm302 constructs, C43(DE3) cells (Lucigen), as an E. coli expression cell line, were transformed by heat shock at 42° C., and then Fm301 and Fm302 expression was confirmed through IPTG induction (FIGS. 7a and 7b). C43(DE3) cells were shaking-cultured in conditions of 37° C. and 150 rpm.

As a result of comparing expression degrees of Fm301 and Fm302 cultured under the same culture conditions, the expression amount of Fm302 was more than that of Fm301. In order to verify whether Fm301 and Fm302 bind to anti-Fab antibody using Fab-specific antibody, purified proteins were subjected to electrophoresis on 12% SDS-PAGE gel, and then the proteins were transferred to the nitrocellulose membrane (NCM), followed by reaction with anti-Fab antibody. As a result, Fm301 was not confirmed, but only Fm302 was confirmed (FIG. 7c).

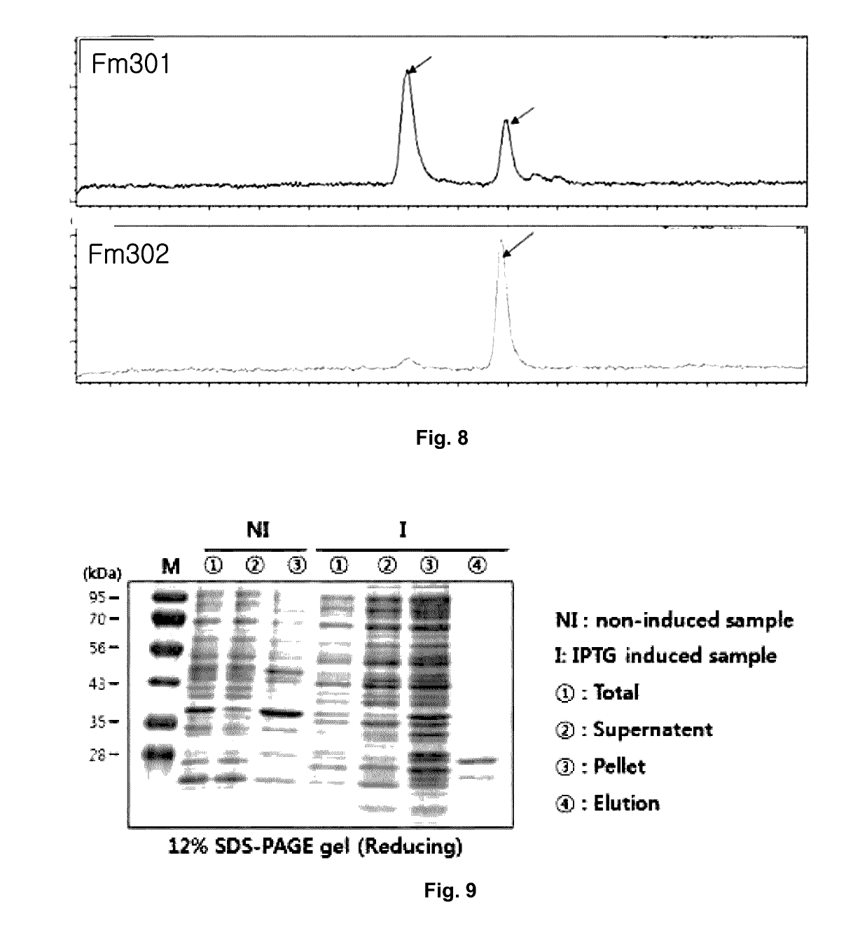

In order to verify the homogeneity of the two antibody fragments, analysis (mobile phase: PBS; column: Bio-sec 2000; flow rate: 1 ml/min; injection dose: 25 μl) of HPLC (SEC2000, Shimadzu, LC-6AD) was conducted, and as a result, Fm302 was confirmed to have higher homogeneity (FIG. 8).

### EXAMPLE 3

### Confirmation on Expression of Fm306 Construct in E. coli

For the expression of Fm306 in E. coli and purification of Fm306, E. coli C43(DE3) cells were used like in Fm302 (under the same conditions as in the protein expression in example 2), the two domains were expressed respectively by using the plasmid pACYCDuet-1 vector, and the antibody fragments were expressed in the periplasm (FIG. 9).

### EXAMPLE 4

### Culture and Purification of Fm302 and Fm306 Antibody Fragments

Culture and Purification of Fm302 Antibody Fragment

E. coli C43(DE3) cells, which can reduce cell death due to toxicity of overexpressed recombinant protein by lowering the level of T7 RNA polymerase, were used as host cells for purification of the antibody fragment. pACYCDuet-1 vector was used as a plasmid, such that two domains were expressed respectively, and the OmpA signal peptide was introduced to generate the antibody fragment in the periplasm.

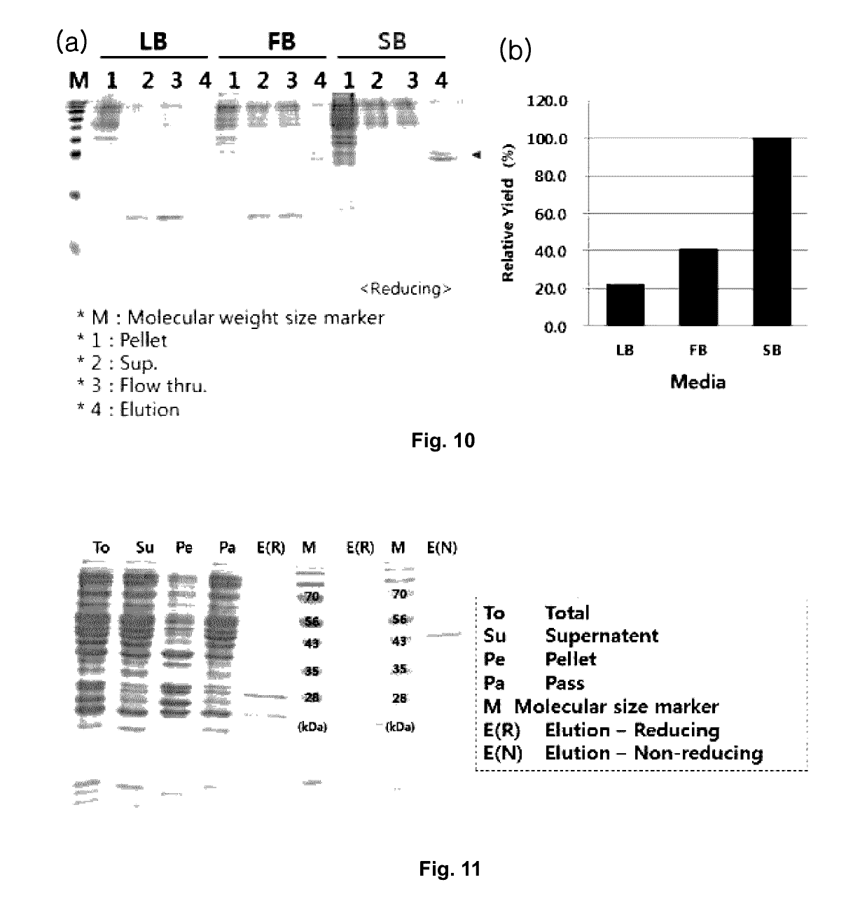

In order to establish optimal culture conditions, the antibody fragment yields were compared for different culture media (FIG. 10a). Lysogeny broth (LB), fastidious broth (FB), and super broth (SB) were used for the comparison test, and the cell mass and the antibody fragment yield were compared under the same culture conditions (37° C. and 150 rpm shaking culture). The final OD₆₀₀ values are 3.69 for LB, 6.58 for FB, and 11.48 for SB, and thus the SB was measured to have the highest value, and this means that the greatest number of cells can be obtained in the SB medium. In the affinity chromatography using KappaSelect resin, it was determined that, as the result of the comparison of relative activity of lane 4 using Fm302 samples, the relative activity in the SB medium was 5-fold higher than that in the LD medium, and this means that the amount of antibody fragment existing in the SB medium would be increased 5-fold compared with that in the LB medium (FIG. 10b).

The antibiotic chloramphenicol was put in the LB medium, and then cultured overnight using a shaking incubator in conditions of 37° C. and 150 rpm. After that, the cell culture medium grown the day before was inoculated into new SBplus medium (Gellix) to have about 1-2%, and the antibiotic chloramphenicol was put therein, followed by culturing using a shaking incubator in conditions of 37° C. and 150 rpm. After the inoculation, the expression was induced using 1 mM IPTG when the OD₆₀₀ value was 3.0, followed by culturing for 9 hours in conditions of 5° C. and 150 rpm. Thereafter, the culture medium was centrifuged in conditions of 4° C. and 8000 rpm, thereby obtaining pellets. A cell lysis buffer [1×phosphate buffered saline (PBS), 5 mM ethylenediaminetetraacetic acid (EDTA), 10% glycerol, pH 7.4] was added to the obtained cells at about 30-40 ml per 1 L of the culture medium, followed by mixing. Thereafter, the cells were lysed using an ultra-sonicator for 5 minutes (pulse on 3 seconds, pulse off 3 seconds). In order to separate the antibody fragment and proteins in an aqueous solution from the cells, centrifugation was conducted using a centrifuge at 20,000 rpm for 40 minutes.

Affinity chromatography was conducted to purify only the antibody fragment from the aqueous solution of antibody fragment and proteins, which was separated from the cells. The open column was filled with KappaSelect resin (GE Healthcare), which binds to the C_{L} domain of the antibody fragment, and an equilibrium buffer (50 mM Tris-HCl, 100 mM NaCl, 5 mM EDTA, pH 7.4) was allowed to flow to make the column an equilibrium state. The aqueous solution of antibody fragment and proteins was allowed to flow so that the resin bound to the antibody fragment, and then 5 column volume (CV) of a washing buffer (50 mM Tris-HCl, 100 mM NaCl, 5 mM EDTA, pH 7.4) was allowed to flow to remove non-specifically bound impurities, In order to separate the antibody fragment from the KappaSelect resin, an elution buffer (100 mM glycine, 1 mM EDTA, pH 2.5) was allowed to flow. As shown in the experimental test, as a result of expressing the Fm302 construct in the pACYCDuet-1 vector, it was confirmed that the antibody fragment was expressed in a soluble type, and was not expressed in an inclusion body type. As a result of primary purification, the purified antibody fragment showed about 80% purity, and as a result of electrophoresis using non-reducing dye, it was confirmed that a light chain and heavy chain heterodimer was formed (FIG. 11).

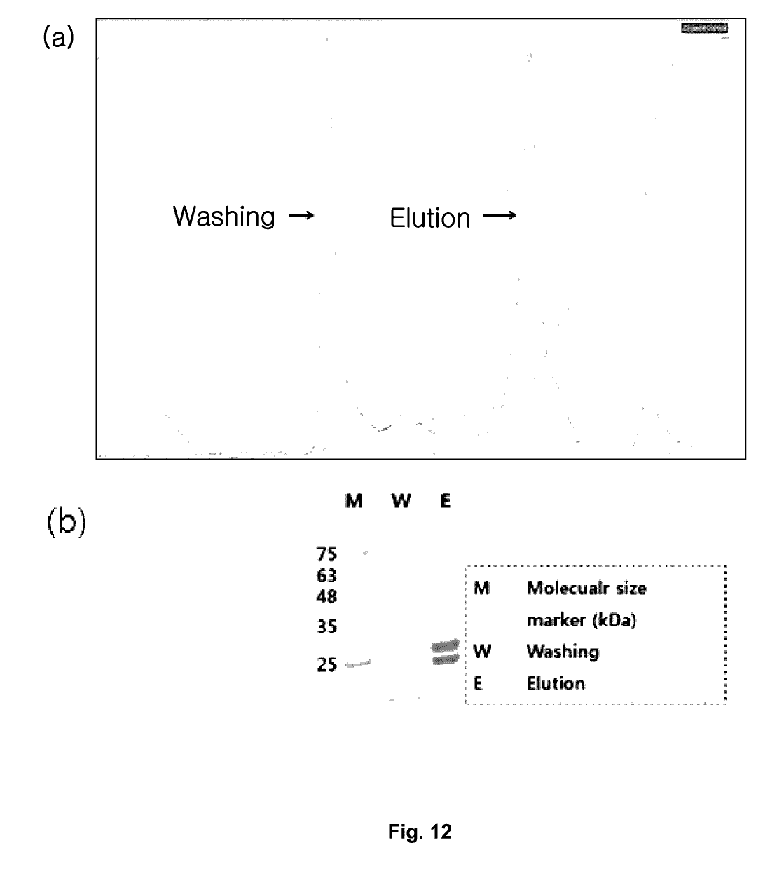

In order to further increase the purity of the primarily purified antibody fragment, ion exchange chromatography was conducted. The HiTrapSP HP column (GE Healthcare) was connected to the AKTA prime FPLC system, and an equilibrium buffer (100 mM glycine, 1 mM EDTA, pH 2.5) was allowed to flow to make the column an equilibrium state. The aqueous solution (pH 2.5) of antibody fragment, which was eluted during the primary purification procedure, was allowed to flow such that the resin bound to the antibody fragment, and then 5 CV of washing buffer 1 (50 mM MES, 1 mM EDTA, 2 mM DTT, pH 6.0) and washing buffer 2 (50 mM MES, 20 mM NaCl, 1 mM EDTA, pH 6.0) were allowed to flow to remove non-specifically bound impurities. In order to separate the antibody fragment from the column, an elution buffer (50 mM MES, 300 mM NaCl, 1 mM EDTA, pH 6.0) was allowed to flow. The SDS-PAGE results of the purified antibody fragment confirmed that impurities with small sizes were all removed through the washing procedure, and the antibody fragment was all eluted during the elution procedure (FIG. 12a). Here, the purity was confirmed to be at least 90% (FIG. 12b).

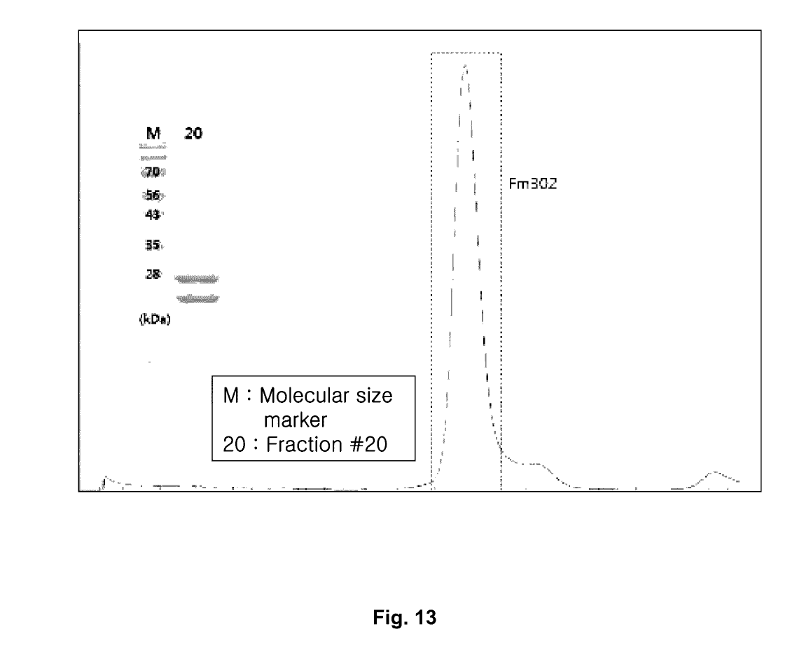

In order to verify the homogeneity of the antibody fragment, size exclusion chromatography was conducted. The HiLoad 161600 Superdex 75 pg column (GE Healthcare) was connected to the AKTA prime FPLC system, and an equilibrium buffer (PBS, 10% glycerol, 1 mM EDTA, 0.02% NaN3, pH 7.2)) was allowed to flow to make the column an equilibrium state. The aqueous solution of antibody fragment, which was eluted during the secondary purification procedure, was allowed to flow, and the retention volume was measured to be 55-65 ml. From 280 nm absorbance results, which show the antibody fragment in chromatography, and SDS-PAGE results, it was thought that the homogeneity of the antibody fragment was very high (FIG. 13).

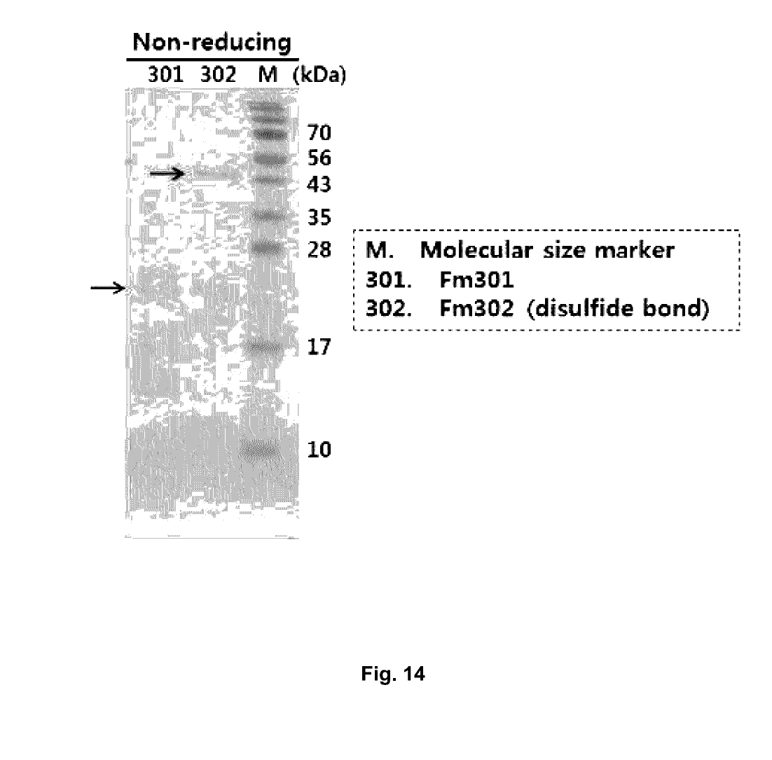

As a result of non-reducing gel electrophoresis analysis of Fm301, which cannot form a disulfide bond due to the absence of Cys at the C-terminal, and Fm302, which can form a disulfide bond, it was confirmed that Fm302 showed one band due to a disulfide bond (FIG. 14).

Culture and Purification of Fm306 Antibody Fragment

For the purification of Fm306, E-coli C43(DE3) cells and pACYCDuet-1 vector, which were the same as those as in Fm302, were used as host cells and a plasmid, and the antibody fragment was generated in the periplasm using the OmpA signal peptide. For seed culture, the antibiotic chloramphenicol was put in the LB medium, followed by culturing overnight using a shaking incubator in conditions of 37° C. and 200 rpm. For main culture, the cell culture medium grown the day before was inoculated into SB+ medium to have about 1-2%, and the biotic chloramphenicol was put therein, followed by culturing using a shaking incubator in conditions of 37° C. and 200 rpm. When 3-4 hours passed after the inoculation, the culture extent was confirmed through absorbance. When the absorbance at OD₆₀₀ was about 1.5, the expression was induced by addition of 1 mM IPTG to the culture medium, followed by culturing overnight in a shaking incubator in conditions of 25° C. and 200 rpm.

Thereafter, the culture medium was centrifuged in conditions of 4° C. and 8000 rpm to obtain precipitates. In order to highly purify the obtained Fm306 antibody fragment, which was expressed in the E-coli C43(DE3) cells, the culture medium was suspended in a cell lysis buffer while about 30-40 ml of the cell lysis buffer was used per 1 L of the culture medium. Specifically, the cells were suspended in a cell lysis buffer of pH 7.4, containing phosphate buffered saline (PBS), 5 mM ethylenediaminetetraacetic acid (EDTA), and 10% glycerol. Thereafter, the cells were lysed using an ultra-sonicator for 5 minutes (pulse on 3 seconds, pulse off 3 seconds). In order to separate the antibody fragment and proteins in an aqueous solution from the cells, centrifugation was conducted using a centrifuge at 20,000 rpm for 40 minutes.

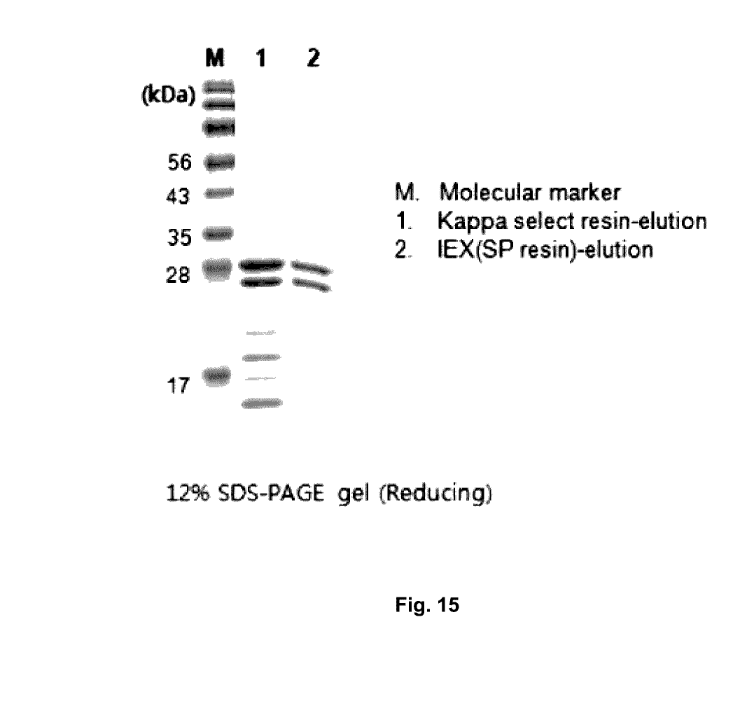

Affinity chromatography was conducted to purify only the antibody fragment from the aqueous solution of antibody fragment and proteins, which was separated from the cells. The open column was filled with KappaSelect resin (GE Healthcare), which binds to the C_{L} domain of the antibody fragment, and an equilibrium buffer (50 mM Tris-HCl, 100 mM NaCl, 5 mM EDTA, pH 7.4) was allowed to flow to make the column an equilibrium state. The aqueous solution of antibody fragment and proteins was allowed to flow such that the resin bound to the antibody fragment, and then 5 column volume (CV) of a washing buffer (50 mM Tris-HCl, 100 mM NaCl, 5 mM EDTA, pH 7.4) was allowed to flow to remove non-specifically bound impurities. In order to separate the antibody fragment from the KappaSelect resin, elution was conducted by an elution buffer (100 mM glycine, 1 mM EDTA, pH 2.5)(FIG. 15, lane 1).

After that, in order to increase purity of the antibody fragment by removing impurities on four bands (FIG. 15, lane 1) around 17 kD in the primarily purified antibody fragments, ion exchange chromatography was conducted for secondary purification, The open column was filled with SP resin (GE Healthcare) of the antibody fragment, and an equilibrium buffer (100 mM glycine, 1 mM EDTA, pH 2.5) was allowed to flow to make the column an equilibrium state. The aqueous solution of antibody fragment and proteins was allowed to flow such that the resin bound to the antibody fragment, and then 5 column volume (CV) of a washing buffer (50 mM MES, 20 mM NaCl, 1 mM EDTA, pH 6.0) was allowed to flow to remove non-specifically bound impurities. An elution buffer (50 mM MES, 300 mM NaCl, 1 mM EDTA, pH 6.0) was allowed to flow to separate the antibody fragment from the column. The purified antibody fragment was confirmed through SDS-PAGE (FIG. 15, lane 2).

SDS-PAGE results confirmed that, as a result of ion exchange chromatography using the SP resin after the primary purification using the KappaSelect resin, the impurities around 17 kD were removed, excluding the target protein, and thus the purity of antibody fragment was increased by at least 90% (FIG. 15).

Preparation of Fm306-PEG Conjugate

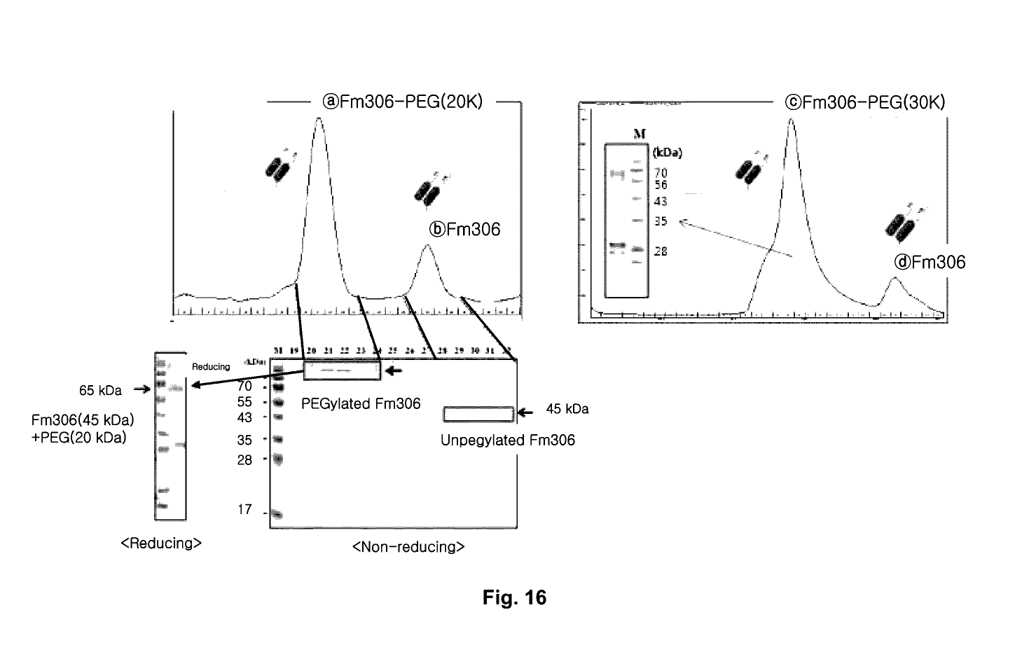

For the pegylation of the Fm306 antibody fragment prepared by the foregoing method, a 1.5 M Tris-Cl buffer was added to a reaction liquid after the purification of Fm306 to adjust pH to about 7.5, and then, fresh PEG-maleimide (NANOCS) was added thereto at a mole ratio of 1:10 immediately before a reaction. Thereafter, the Fm306-PEG mixture solution was mixed on a stirrer at room temperature. The reaction time was 2 hours. After the reaction, the SP resin was used to remove free-PEG not binding to the antibody fragment. The pH of the Fm306-PEG mixture solution was lowered to about 2.5 by using a HCl buffer, and then the Fm306-PEG mixture solution was allowed to bind to the SP resin. Thereafter, washing was conducted using 20 CV or more of a buffer having the same pH (100 mM Glycine pH 2.5, 1 mM EDTA), thereby removing free-PEG remaining without binding to the antibody fragment and obtaining the Fm306-PEG conjugate. This conjugate was loaded on a Superdex 200 column (GE Healthcare) equilibrated with a 10 mM phosphate buffered saline (PBS, pH 7.3), and the conjugate was eluted from the column at a flow rate of 1 ml/min using the same buffer. The Fm306-PEG conjugate (\textcircled{a} of FIG. 16) has a relatively larger molecular weight than the Fm306 (\textcircled{b} of FIG. 16), and thus was first eluted, and therefore the Fm306-PEG conjugate was separated by using such a feature. Respective fractions were confirmed on SDS-PAGE non-reducing gels, such that the Fm306-PEG conjugate showed a size of around 100 kDa or more and Fm306 showed a size of 45 kDa. It is known that, generally, pegylated proteins slowly move on PAGE, and thus it is difficult to show their sizes (Anal Biochem. 1992 Feb. 1; 200(2):244-8). As a result of confirming this fact using reducing gels, proteins were confirmed at about 65 kDa and 28 kDa, which are considered to be pegylated antibody fragment (65 kDa) and a light chain region of the Fm306 antibody fragment. The light chain region remaining without pegylation due to the site-specific pegylation of the heavy chain was confirmed. On the SDS-PAGE of Fm306-PEG(30K), the heavy chain was shown at 70 kDa, which was calculated as around 85 kDa corresponding to two reacting PEG molecules with 30 kDa, and when considering that the proteins shows a trend of smaller sizes, the protein was considered to be an antibody fragment in which one PEG molecule was homogeneously pegylated through site-specific pegylation. The yield of the thus obtained Fm306-PEG(20K) was confirmed to be about 80%. The next experiment was conducted on the pegylated antibody fragment (FIG. 16).

The Fm306-PEG(30K) conjugate was also prepared using the same method as in the Fm306-PEG(20K) conjugate.

The Fm306-PEG conjugate (\textcircled{c} of FIG. 16) has a relatively larger molecular weight than the Fm306 (\textcircled{d} of FIG. 16), and thus was first eluted, and therefore the Fm306-PEG conjugate was separated by using such a feature. As a result of confirming this fact using reducing gels, two bands were confirmed at about 70 kDa and 28 kDa, which are considered to be a pegylated antibody fragment (75 kDa) and heavy and light chain regions of the Fm306 antibody fragment. Like in Fm306-PEG(20K), the light chain region remaining without pegylation due to the site-specific pegylation of the heavy chain region was confirmed, and for the same reason, the protein was considered to be an antibody fragment in which one PEG molecule was homogeneously pegylated. The yield of the thus obtained Fm306-PEG(30K) was confirmed to be about 80%. The next experiment was conducted on the pegylated antibody fragment (FIG. 16).

Selection of Fab′ Construct

In order to specifically introduce PEG to the C-terminal of the C_{H1} domain of the antibody fragment Fab′, Fm306, in which six amino acid sequences (THTCAA) were added to the C-terminal of the C_{H1} domain of Fm302, was selected as a pegylation construct. The pegylation was made through a reaction between a mercapto (sulfhydryl) functional group of the cysteine residue among the added amino acid sequence and the maleimide functional group located at the terminal of PEG. In order to demonstrate that only the cysteine of the amino acid sequence for pegylation, which was added to the C-terminal of the C_{H1} domain of Fm306, is pegylated but cysteine residues existing at the other sites of Fm306 are not pegylated, a comparison experiment between the Fm301 construct and the Fm302 construct was conducted. Fm301, which is a construct without cysteine at the C-terminal thereof, was used as a control construct for demonstrating that the cysteine residues constituting an intra-chain disulfide bond were not pegylated. Fm302, which is a construct with cysteine at the C-terminal of each chain thereof, was selected to demonstrate that the corresponding cysteine residues were not pegylated (FIG. 17).

Preparation of Pegylated Fab′

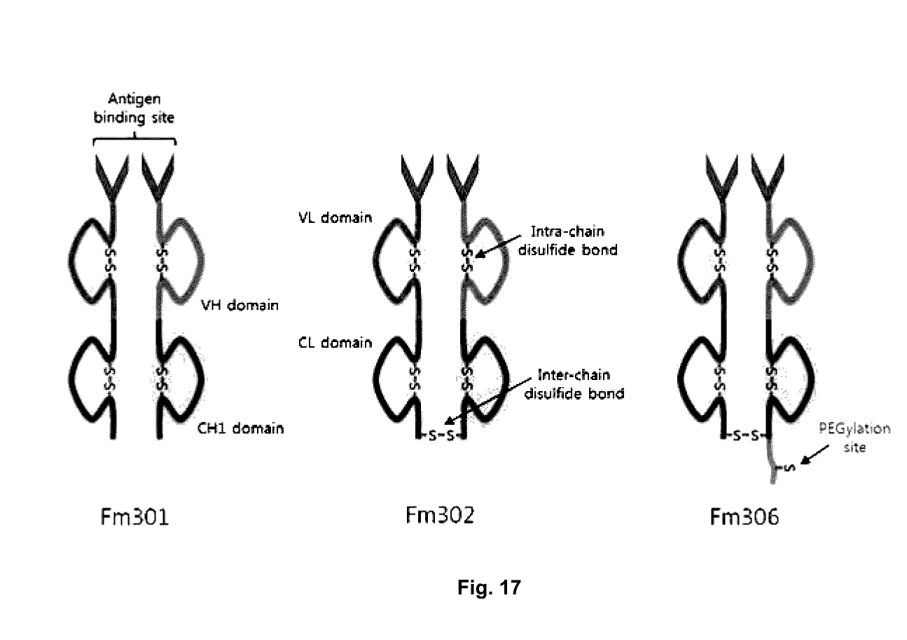

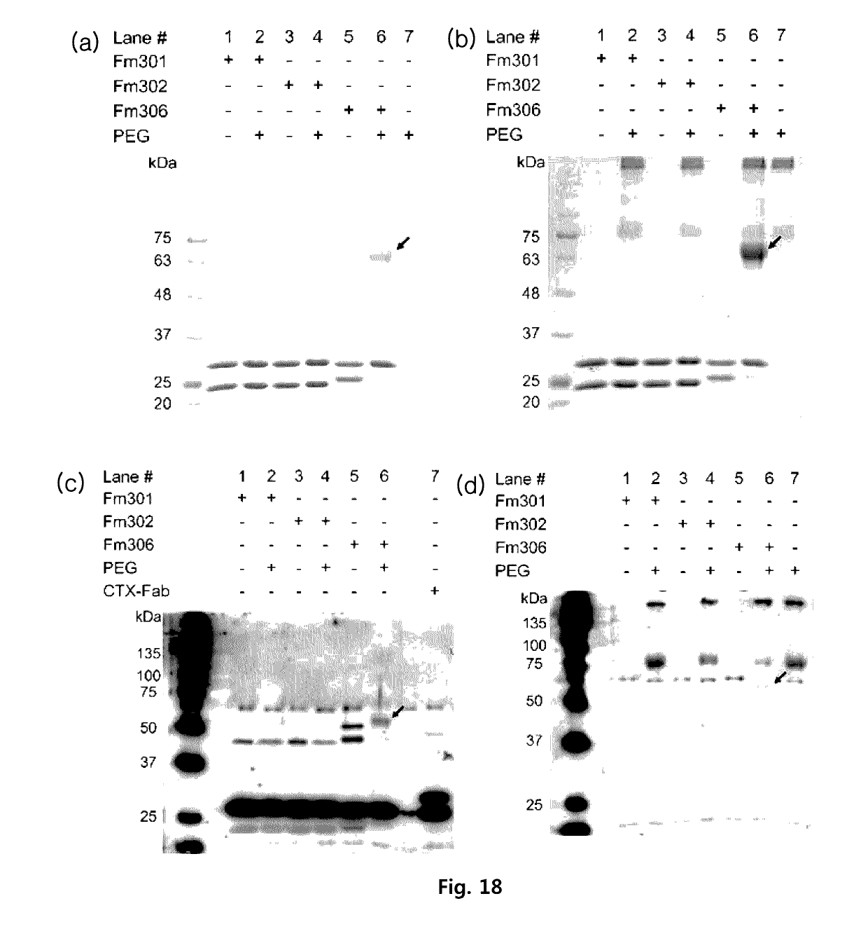

For the preparation of pegylated Fm306 (Fm306-PEG), a 1.5 M Tris-HCl buffer was added to the reaction liquid to adjust the pH to around 7.5, after Fm306 purification, and then PEG-maleimide was mixed therewith such that the ratio of Fm306 and PEG-maleimide was 1:10. Thereafter, a reaction was conducted using a stirrer at room temperature for 2 hours. Fm301 and Fm302 were also purified by the above same method, and then a pegylation reaction was performed by adding PEG. In order to verify whether only Fm306, as a pegylation construct, was specifically pegylated, SDS-PAGE was conducted (FIG. 18a). To find out whether pegylation occurred, antibody fragment samples not reacting with PEG (lanes 1, 3, and 5 in FIG. 18a) and samples of which pegylation was attempted by adding PEG (lanes 2, 4, and 6 in FIG. 18b), for the respective constructs, were prepared, and then were loaded on reducing gels. In the antibody fragment samples, such as Fm301, Fm302, and Fm306 (lanes 1, 3, and 5 in FIG. 2a) and Fm301 and Fm302 samples subjected to an attempt to react with PEG (lanes 2 and 4 in FIG. 18a), the light chain regions and the heavy chain regions of the antibody fragment samples were confirmed at the about 25-kDa position, but in Fm306 sample reacting with PEG (lane 6 in FIG. 18a), a pegylated heavy chain region of about 65 kDa and a light chain region of 25 kDa were confirmed. This result demonstrates that the cysteine of the amino acids added to the terminal of the domain was specifically pegylated. Also in the Fm301 and Fm302 samples subjected to an attempt to react with PEG (lanes 2 and 4 in FIG. 18b), the presence of PEG molecules was confirmed through PEG staining. However, the PEG molecule was not shown at the same position as in the antibody fragments, but was shown at the same position as in the PEG sample (lane 7 in FIG. 18b). This means that the PEG molecule did not bind to the antibody fragments, and thus demonstrates that the cysteine residues forming the intra-chain disulfide bonds and the inter-chain disulfide bonds present in the antibody fragments were not pegylated. Western blot analysis was conducted on the same samples by using an anti-Fab antibody specifically binding to the antibody fragments and an anti-PEG antibody specifically binding to PEG. When the Fab specific antibody and the PEG specific antibody were used, the antibody fragment (lane 6 in FIG. 18c) and PEG (lane 6 in FIG. 18d) were confirmed at the same position on SDS-PAGE.

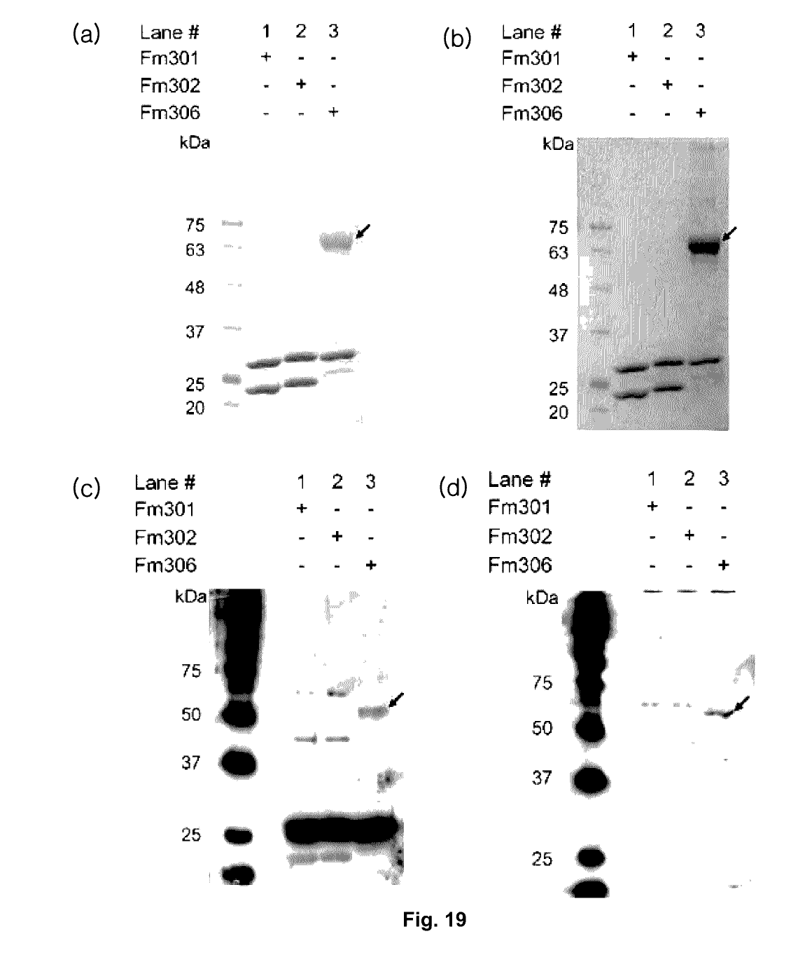

After the reaction, in order to remove residual PEG not binding to the antibody fragment, the Fm306-PEG mixture solution was loaded on the column filled with KappaSelect resin. Thereafter, 20 CV or more of a buffer (50 mM Tris-HCl, pH 8.0, 100 mM NaCl, and 5 mM EDTA) was allowed to flow to remove residual PEG through sufficient washing, followed by elution using a buffer (100 mM Glycine, pH 2.5, 1 mM EDTA). The eluted Fm306-PEG was loaded on the Superdex 200 column (GE Healthcare) equilibrated with a 10 mM phosphate buffered saline (PBS, pH 7.4), and then eluted from the column at a flow rate of 1 ml/min using the same buffer. Also for Fm301 and Fm302, residual PEG was removed by the same method. As a result of observing the eluted samples on reducing gels using SDS-PAGE, the heavy chain region binding to PEG was confirmed at the about 65-kDa position in Fm306 (lane 3 in FIG. 19a), and antibody fragments binding to PEG were not shown in Fm301 and Fm302 (lanes 1 and 2 in FIG. 19a), Similarly to the above, PEG was confirmed at the same position in only the Fm306 sample (lane 3 in FIG. 3), and PEG was not confirmed in the Fm301 and Fm302 samples (lanes 1 and 2 in FIG. 19b). This means that, in both Fm301 and Fm302, the antibody fragments were all removed without binding to PEG during the purification procedure. The western blot analysis was conducted using the same method described above, and the antibody fragment (lane 3 in FIG. 19c) and PEG (lane 3 in FIG. 19d) were confirmed at the same position.

According to the above results, the antibody fragment bound to PEG in only the Fm306 construct, and the antibody fragments did not bind to PEG in Fm301 and Fm302. Therefore, it was confirmed that the site-specific pegylation occurred at the only cysteine of the amino acid sequence (THTCAA) introduced to Fm306, and the pegylation did not occur at the cysteines that form the intra-chain disulfide bond of Fm301 and the intra-chain disulfide bonds and the inter-chain disulfide bonds of Fm302 and Fm306. In addition, for Fm306-PEG (lane 6 in FIG. 18a and lane 3 in FIG. 19a), only one band corresponding to the heavy chain region was confirmed, and this demonstrates monomeric pegylation. Therefore, the site-specific pegylation and the monomeric pegylation were confirmed through the foregoing methods.

Confirmation on Purified Antibody Fragment Through EDMAN Sequencing

In order to verify purified antibody fragment and the deletion or not of the signal peptide of the antibody fragment, EDMAN sequencing was requested at the eMass analysis Lab to verify the purified antibody fragments and the removal of the signal peptide at the N-terminal. As shown in table 17, the N-terminal sequences of the antibody fragment were confirmed to be D-I-L-L-T for the light chain and Q-V-Q-L-K for the heavy chain.

TABLE 17

| | | |
|---|---|---|
| Fm302 light chain | Analysis result | D-I-L-L-T |
| Fm302 heavy chain | | Q-V-Q-L-K |

### EXAMPLE 5

### Measurement of Antigen-Binding Activity of Purified Antibody Fragment

The western blot assay using CH3 antibody was conducted to verify whether the purified protein was an antibody fragment, and the binding affinity was measured using SPR and ELISA to verify whether the purified protein has an ability to bind to antigen EGFR.

Measurement of sEGFR-Binding Affinity Using Surface Plasmon Resonance (SPR)

Three types of antibody fragments containing a positive control (cetuximab) were coated on XPR GLM chip, and EGFR-Fc was allowed to flow therethrough to verify the non-specific binding or not, and then the optimal binding conditions were confirmed. Based on this, the binding affinity was evaluated by again coating one chip with cetuximab and antibody fragments in the same manner and then allowing EGFR-Fc to flow therethrough. For measurement, the GLM chip of the ProteOn XPR36 system was initialized using 50% glycerol, and then a running buffer (PBS containing 10 mM Na-phosphate, 150 mM NaCl, and 0.005% Tween 20, PBST, pH 7.4) was allowed to flow under 25° C. conditions to make chip stabilization. 220 of 1:1 of 0.04 M N-ethyl-N′-(3-dimethylaminopropyl) carbodiimide (EDC) and 0.001 M sulfo-N hydroxysuccinimide (sulfa-NHS) were allowed to flow through four channels of the GLM chip at a flow rate of 30 μl/min, thereby activating the channels, Cetuximab and antibody fragments of 100 nM each were coated at a rate of 30 μl/min using an acetate buffer of pH 5.5. Then, the activated chip was deactivated with 1 M Ethanolamine-HCl (pH 8.5), it was confirmed that the immobilization levels thereof were 605-4340 resonance units (RU), respectively. The KD value was calculated by setting a reference using PBST, making a series of five concentrations obtained through ½-fold dilution of 5-50 nM EGFR-Fc, and allowing the diluted EGFR-Fc to flow through the coated portions. The zero base was confirmed using a regeneration buffer (50 mM NaOH), and then the final result value was deduced under optimal conditions through repeated tests.

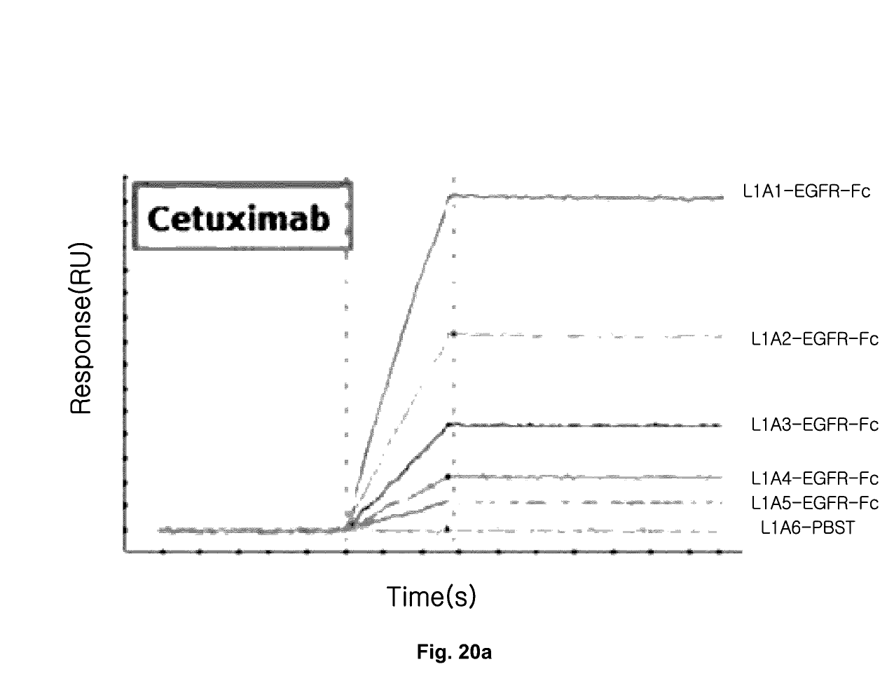

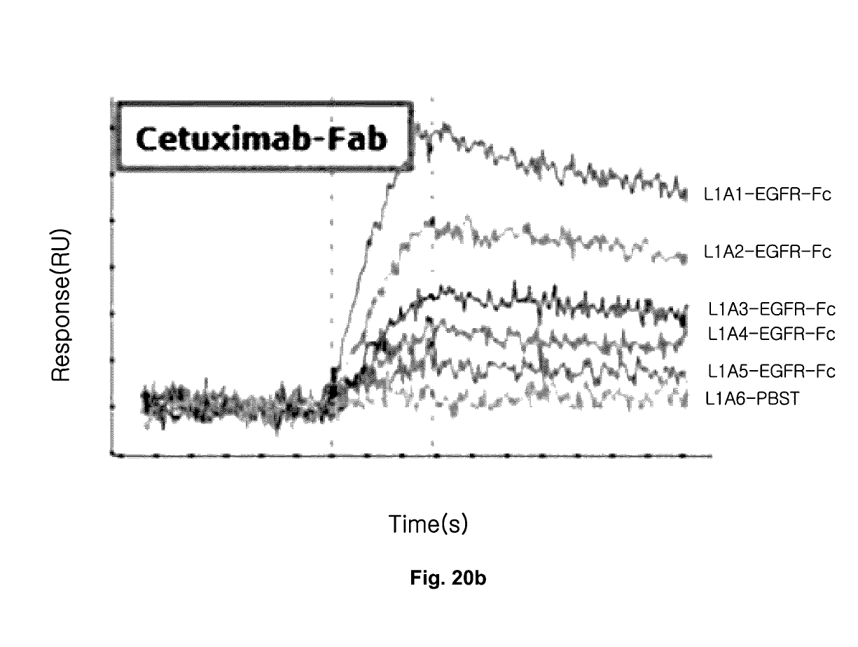

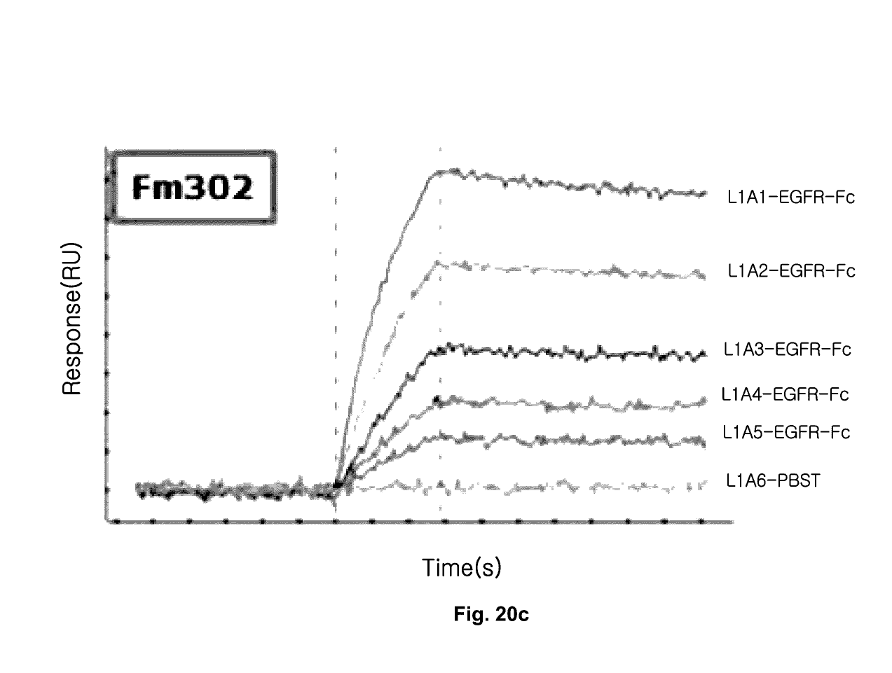

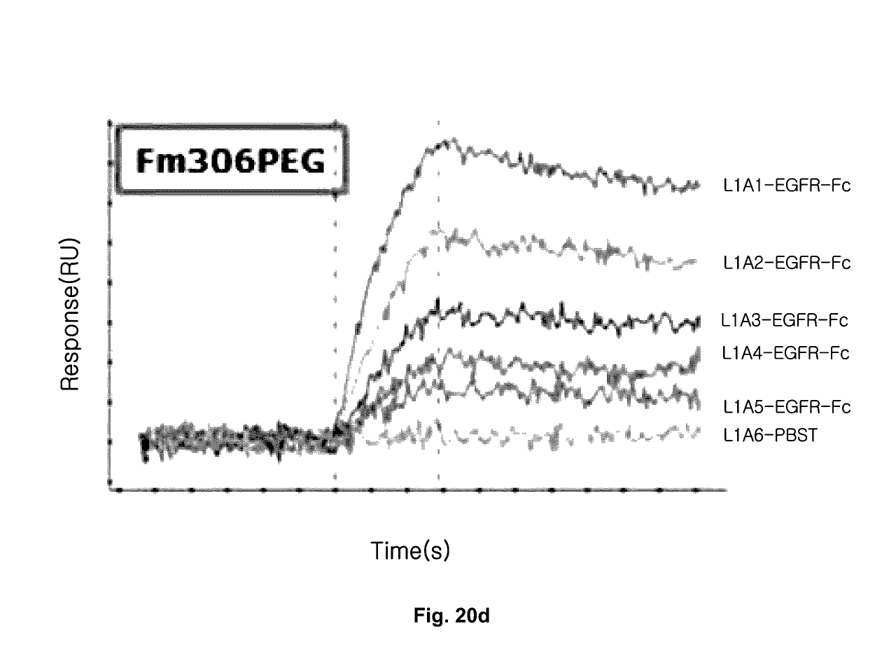

When cetuximab and the antibody fragments were coated on one chip in the same manner and the same concentration of EGFR was allowed to flow on the chip, the binding degree of cetuximab to EGFR is different from that of the antibody fragments in view of sensitivity, and thus for cetuximab, the kinetic value was obtained starting from 5 nM of EGFR-Fc and, for the other antibody fragments, the optimal conditions were found out starting from 50 nM of EGFR. The test was repeated five times or more to calculate average values. As a result, the kD values were confirmed to be 17.5 pM for cetuximab, 3.45 nM for cetuximab-Fab, 720 pM for FM302, and 1.84 nM for FM302-GPC (table 18 and FIGS. 20a and 20b). The kD value of Fm302 was measured to be somewhat higher compared with cetuximab. However, from the fact that the kD value of Fm302 was measured to be lower than that of cetuximab-Fab, the binding affinity of Fm302 to EGFR is determined to be sufficiently high. In addition, the kD value of Fm306PEG was measured to be lower than that of cetuximab-Fab, and this result means that the reduction in the binding affinity due to pegylation was not great.

TABLE 18

| Classi- | | | | | |
|---|---|---|---|---|---|
| fication | ka(M⁻¹s⁻¹) | kd(s⁻¹) | KD(M) | Rmax | Chi² |
| Cetuximab | 8.90 × 10⁵ | 1.56 × 10⁻⁵ | 1.75 × 10⁻¹¹ | 606.73 | 4.72 |
| Cetuximab- | 2.26 × 10⁵ | 7.78 × 10⁻⁴ | 3.45 × 10⁻⁹ | 36.43 | 2.02 |
| Fab | | | | | |
| Fm302 | 2.60 × 10⁵ | 1.87 × 10⁻⁴ | 7.20 × 10⁻¹⁰ | 96.42 | 3.79 |
| Fm306PEG | 2.41 × 10⁵ | 4.42 × 10⁻⁴ | 1.84 × 10⁻⁹ | 43.91 | 2.02 |

Measurement of sEGFR-Binding Affinity Using ELISA

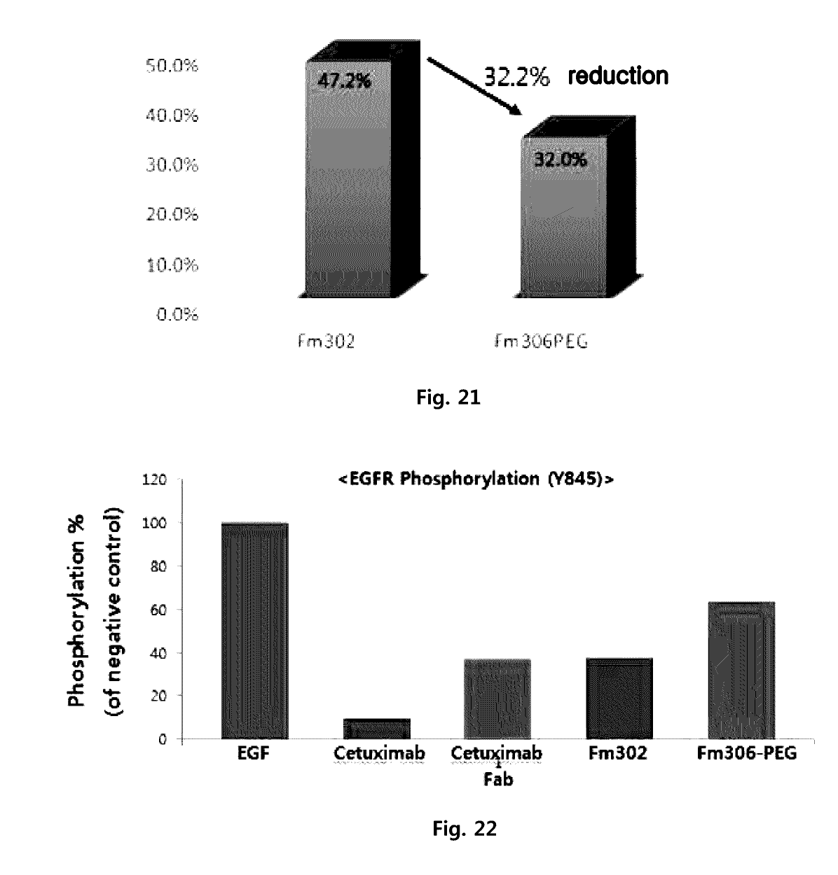

Antigen sEGFR (100 ng/well) was put in the 96-well ELISA plate, followed by reaction at 4° C. overnight, so that the antigen was immobilized on the plate surface. After that, supernatant was removed, and 200 μl of a blocking solution (Sigma, B6429-500ML) was dispensed to each well, followed by blocking at 4° C. overnight. Cetuximab as a standard material for obtaining a calibration curve and the purified antibody fragments were diluted to 0-125 ng/ml using PBS. Each of the diluted solutions was dispensed at 100 μl, followed by reaction at room temperature for 1 hour, thereby inducing the binding with antigen. Upon the completion of the reaction, washing using PBST (PBS, 0.05% tween 20, pH 7.4) was conducted three times. Anti-human IgG (Sigma, 15260) was diluted to 1/1,000, and then dispensed at 100 μl per well, followed by reaction at room temperature for 1 hour. Then, the supernatant was removed, followed by washing with PBST three times. Secondary antibody anti-goat IgG-peroxidase (Sigma, A5420) was diluted to 1/3,000 fold, and then dispensed at 100 μl per well, followed by reaction at room temperature for 1 hour. The supernatant was removed, followed by washing with PBST three times, and then TMB (coloring reagent) was dispensed at 100 μl each. 100 μl of 1 M H₂SO₄ was put in each of the colored wells to stop the reaction, and the absorbance at 450 nm was measured using a microplate reader. The binding affinity of Fm302 was measured to be about 47.2% compared with cetuximab, and this result means that the activity of the antibody fragment to EGFR was sufficiently maintained. In addition, the binding affinity of Fm306PEG was reduced by about 32.2% compared with Fm302, and from this result, the reduction in binding affinity of Fm306PEG due to pegylation was not great, resulting in maintaining about 70% activity (FIG. 21).

Confirmation of EGFR Phosphorylation of Purified Fab in A431 Cell Line

A431 cells under culturing were treated with 0.25% trypsin/EDTA to isolate single cells from each other. The cells were inoculated at 1×10⁶ cells on culturing dishes, followed by stabilization for 24 hours, Thereafter, the cells were cultured in media not containing serum for 8 hours, and then treated with 30 of antibody and antibody fragments, New media were exchanged for the cells under culturing, and then the cells were recovered from the culture dishes, followed the method represented by the Pathscan total EGF receptor sandwich ELISA kit by the cell signaling company.

It was confirmed that, when EGFR-overexpressed A431 cells were treated with antibody fragments, EGFR phosphorylation was blocked by the antibodies, thereby suppressing the binding of phospho-EGF receptor (Try845), and cetuximab-derived cetuximab Fab and Fm302 were confirmed to have the phosphorylation inhibitory ability of about 35-40% at 30 μg/ml (FIG. 22).

### EXAMPLE 6

### Confirmation on Efficacies of Fm302 and Fm306-PEG in Disease Animal Model

The anticancer efficacy of the purified antibody fragment Fab was confirmed in the head and neck cancer disease animal model. In order to prepare the head and neck cancer disease animal model, nude mice, which are deficient in immunity since T cells related to immune functions are not generated due to the lack of thymus, were subcutaneously administered with 1×10⁷ cancer cells obtained by culturing A431 cells, thereby preparing the human tumor xenograft disease model. When tumor tissues were generated and grown to a size of about 50-100 mm′, the administration of antibody fragments and a drug was started, The drug was intravenously administered at 0.25 mg per mouse twice a week. Overall dosing was conducted six times for three weeks. The size of the tumor tissue was measured before drug administration twice a week.

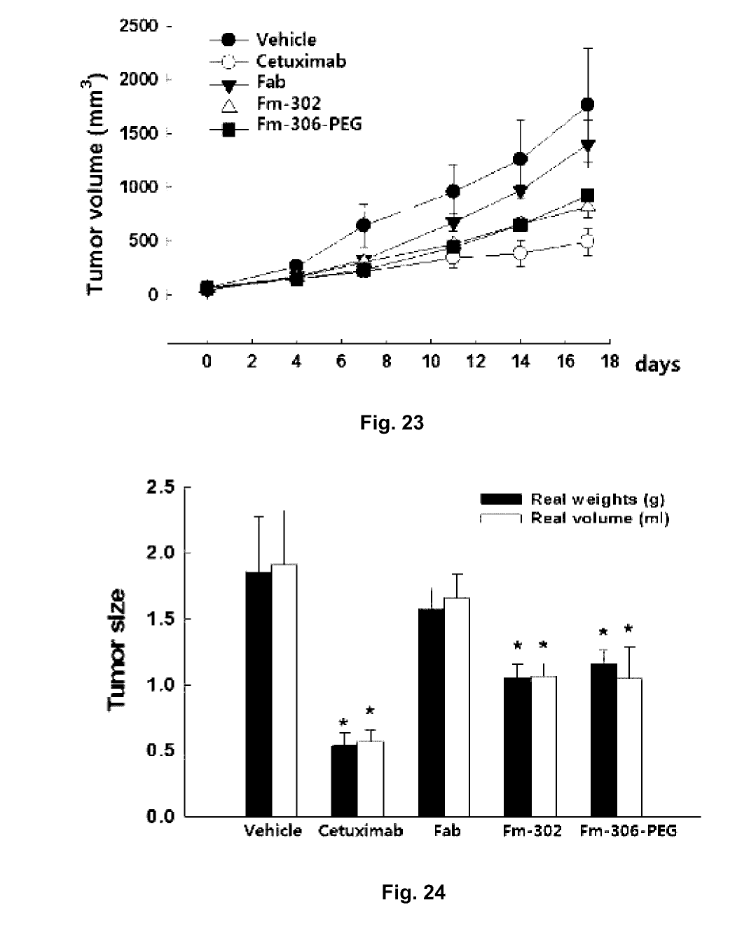

It was confirmed that, when the tumor tissues of the disease animal model were treated with 0.25 mg per mouse twice a week, the tumor growth inhibitory ability of Fm302 was lower than that of cetuximab but was higher than that of cetuximab Fab, and Fm306-PEG had higher tumor growth inhibitory growth than cetuximab Fab due to the increase in half-life, in the third week (FIG. 23).

At the end of the experiment of the head and neck cancer disease model, autopsy was conducted, and the tumor tissues were weighed. As a result, the weight of the tumor tissues was confirmed to have a similar trend to the tumor growth curve (FIG. 24).

Pharmacokinetic Analysis of Purified Antibody Fragments

Experimental animals were intravenously administered with purified antibody fragments at 0.25 mg per mouse through tails thereof, and then the blood was collected from the retro-orbital venous plexus according to time. The collected blood was centrifuged at 3000 rpm for 10 minutes to separate plasma, and then the amounts of antibody fragments present in the separated plasma were measured.

As a result of measuring concentrations of antibody fragments in the blood, it was confirmed that the half-lives of the blood concentration of Fab and Fm302 were within 4 hour after administration, and the half-life of the blood concentration of Fm306 was increased five-fold or more compared with before Fm306 was pegylated. In addition, it was confirmed that the half-life of the blood concentration was increased from 18, 6 h for Fm306-PEG(20kD) to 28.31 h for Fm306-PEG(30kD) (table 19).

TABLE 19

| | | | | PEGylated | PEGylated |
|---|---|---|---|---|---|
| | Cetuximab | Fab | Fm302 | Fm306 (20 KDa) | Fm306 (30 kDa) |
| Cmax | 538.5 ± 131.9 | 209.9 ± 112.5 | 239.4 ± 141.8 | 169.0 ± 62.6 | 371.77 ± 32.21 |
| (μg/mL) | | | | | |
| AUC | 15051.1 ± 4052.2 | 67.8 ± 33.6 | 83.4 ± 51.6 | 1634.2 ± 586.5 | 5057.17 ± 1023.29 |
| (μg · h/mL) | | | | | |
| T½(h) | 89.8 ± 12.5 | 4.1 ± 0.6 | 4.2 ± 0.6 | 18.6 ± 2.0 | 28.31 ± 6.11 |

| | |
|---|---|
| Tmax | After injection (iv, 0.25 mg/mice) |

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

What is claimed is:

1. A fragment antigen-binding (Fab) fragment specifically binding to epidermal growth factor receptor (EGFR), the Fab fragment comprising:
(a) a heavy chain variable region (V_{H}) comprising an amino acid sequence of SEQ ID NO: 4;
(b) a heavy chain variable region 1 (C_{H1}) comprising an amino acid sequence of SEQ ID NO: 5;
(a) a light chain variable region (V_{L}) comprising an amino acid sequence of SEQ ID NO: 6; and
(d) a light chain constant region (CO comprising an amino acid sequence of SEQ ID NO: 7.

2. The Fab fragment of claim 1, wherein the C_{H1} further comprises Cys-Asp-Lys at the C-terminal thereof.

3. The Fab fragment of claim 1, wherein the C_{L} further comprises Glu-Cys at the C-terminal thereof.

4. The Fab fragment of claim 2, wherein the C_{H1} has Thr-His-Thr-Cys-Ala-Ala further linked to Cys-Asp-Lys at the C-terminal thereof.

5. The Fab fragment of claim 1, wherein the Fab fragment is pegylated.

6. The Fab fragment of claim 5, wherein the C_{H1} of the Fab fragment is pegylated.

7. The Fab fragment of claim 6, wherein in the Thr-His-Thr-Cys-Ala-Ala at the C-terminal of the C_{H1} of the Fab fragment, the Cys residue is pegylated.

8. The Fab fragment of claim 5, wherein the polyethylene glycol (PEG) used in the pegylation has a molecular weight of 5-50 kDa.

9. The Fab fragment of claim 8, wherein the PEG has a molecular weight of 18-25 kDa.

10. The Fab fragment of claim 2, wherein the V_{L} further comprises Glu-Cys at the C-terminal thereof, and wherein the Cys residue of the Cys-Asp-Lys at the C-terminal of the C_{H1} is linked to the Cys residue of the Glu-Cys at the C-terminal of the V_{L} via a disulfide bond.

11. The Fab fragment of claim 1, wherein the half-life of the Fab fragment in mice (Mus musculus) is 20-35 hours.

12. An expression construct for preparing a fragment antigen-binding (Fab) fragment specifically binding to epidermal growth factor receptor (EGFR), the expression construct comprising:
(a) a heavy chain-expression construct comprising: (a-1) a heavy chain variable region (V_{H})-encoding nucleic acid molecule comprising a nucleotide sequence of SEQ ID NO: 9; and (a-2) a heavy chain constant region 1 (C_{H1})-encoding nucleic acid molecule comprising a nucleotide sequence of SEQ ID NO: 10; and
(b) a light chain-expression construct comprising: (b-1) a light chain variable region (V_{L})-encoding nucleic acid molecule comprising a nucleotide sequence of SEQ ID NO: 11; and (b-2) a light chain constant region (C_{L})-encoding nucleic acid molecule comprising a nucleotide sequence of SEQ ID NO: 12.

13. A recombinant vector comprising the expression construct of claim 12.

14. A host cell transformed with the recombinant vector of claim 13.

15. The host cell of claim 14, wherein the host cell is E. coli.

16. A method for preparing a fragment antigen-binding (Fab) fragment specifically binding epidermal growth factor receptor (EGFR), the method comprising:
(a) culturing the host cells of claim 14; and
(b) expressing the Fab fragment to EGFR in the host cells.

17. A pharmaceutical composition for preventing or treating cancer, comprising: (a) a pharmaceutically effective amount of the fragment antigen-binding (Fab) fragment specifically binding to epidermal growth factor receptor (EGFR) of claim 1; and (b) a pharmaceutically acceptable carrier.

18. The pharmaceutical composition of claim 17, wherein the cancer is breast cancer, large intestine cancer, lung cancer, stomach cancer, liver cancer, blood cancer, bone cancer, pancreatic cancer, skin cancer, brain cancer, cervical cancer, nasopharyngeal cancer, laryngeal cancer, colorectal cancer, ovarian cancer, rectal cancer, large intestine cancer, vaginal cancer, small intestine cancer, endocrine cancer, thyroid cancer, parathyroid cancer, ureter cancer, urinary tract cancer, prostate cancer, bronchial cancer, bladder cancer, kidney cancer, or bone marrow cancer.
